(19) United States
(12) Patent Application Publication
(10) Pub. No.: US 2025/0059246 A1
(43) Pub. Date: Feb. 20, 2025

(54) MODIFIED DAIRY PROTEINS, METHODS FOR THEIR PRODUCTION AND USE

(71) Applicant: MIRUKU LIMITED, Palmerston North (NZ)

(72) Inventors: Alejandra ACEVEDO FANI, Palmerston North (NZ); Harjinder SINGH, Palmerston North (NZ); Mai Shamir ASOR, Ma'ale Gamla (IL); Oded SHOSEYOV, Shoham (IL); Miron ABRAMSON, Har Adar (IL)

(21) Appl. No.: 18/723,331

(22) PCT Filed: Dec. 21, 2022
(86) PCT No.: PCT/IB2022/062639

§ 371 Date: Jun. 21, 2024

(30) Foreign Application Priority Data
Dec. 24, 2021 (AU) 2021904275

(51) Int. Cl. C07K 14/47; A23C 11/08

(52) U.S. Cl. CPC C07K 14/4732; A23C 11/08

(57) ABSTRACT

The invention provides modified dairy proteins in which at least one potentially phosphorylated amino acid has been replaced with a negatively charged amino acid. The modified dairy proteins exhibit at least one of: an improved capacity to binding calcium, an improved capacity to form a micelle, an altered isoelectric point (pl), altered thermal stability and an altered zeta potential, when expressed in a heterologous expression system, relative to that of the unmodified dairy protein when it is recombinantly expressed in the same heterologous expression system. The invention also provides cells, tissues, plants, plant parts and seeds, expressing the modified dairy proteins. The invention also provides methods for the production and use of the modified proteins. The invention also provides food or beverage products or ingredients comprising the modified dairy proteins.

### CROSS REFERENCE TO RELATED APPLICATIONS

The contents of Australian provisional patent application number 2021904275, filed 24 in December 2021, is incorporated herein by reference in its entirety.

### FIELD OF INVENTION

The invention is in the field of modified proteins, recombinantly produced host cells, plant cells and plants, and use of the proteins, particularly in the food and beverage industry.

### BACKGROUND TO THE INVENTION

Sustainable and healthy supply of protein is one of defining challenges of our time. One of the key issues is how we diversify from animal protein to more sustainable plant and other alternative proteins. This is mainly driven by consumer perceptions around nutrition, health, animal welfare, the sustainability of food production, and food security. This protein supply diversification process needs to make high quality proteins more available to humans, either by turning plant proteins into desirable food products (that resemble animal-based product functionality and nutritional quality) or by partly substituting plant proteins for animal proteins or producing animal proteins without the use of animals (e.g. through recombinant technologies, in-vitro farming). These options will provide dietary protein for the growing population with a lower environmental footprint and an affordable cost point.

There is strong interest in recombinantly produced dairy proteins. However, in order to increase consumer acceptance of these animal-free dairy proteins, these proteins must be able to mimic the taste, smell, sensations, physical and nutritional functionality of milk and milk products.

Casein and whey protein fractions may be separated and isolated from milk at an industrial scale. These include caseins and caseinates, whey protein concentrates and isolates, milk protein concentrates and isolates, and hydrolysed proteins. These protein ingredients possess functional properties which provide desirable textural or other attributes to the final product and for this reason have found numerous applications in traditional dairy products and in other foods.

For example, caseins are amphiphilic molecules with a well-balanced distribution of hydrophilic and hydrophobic domains which allows them to be used in food products where incorporation of oil/water/air into the continuous system is required, such as medical and nutritional beverages, imitation cheeses, salad dressings, mayonnaise, liqueurs, meringues, soufflés, whipped toppings, confectionary, sponge cakes, etc. Because of their ability to form rigid, heat-induced irreversible gels, whey protein products have found many applications in food products, including meat products, dairy desserts, sausages, bread and cakes for example.

Efforts have been made to express such milk proteins in heterologous systems. For example, to date, much attention has been focused on the production of recombinant β-lactoglobulin through its expression in E. coli, yeast and in Trichoderma reesei for use in food products by companies such as Perfect Day Inc.

Similarly, the production of recombinant proteins based on animal protein using transgenic plants is known, for example in the production of dairy proteins. However, the suitability of these known recombinant proteins in the production of final food products that mimic animal based dairy proteins in functionality, flavour and “experience” is unclear. The use of known approaches may therefore produce recombinant proteins having less than desirable characteristics for use as a dairy substitute in food and beverage production.

For example, the phosphorylation of casein does not occur in transgenic plants expressing recombinant beta-casein (Philip et al., 2001, Processing and localization of bovine β-casein expressed in transgenic soybean seeds under control of a soybean lectin expression cassette. Plant Science, 161 (2), pp. 323-335). Given that phosphorylation plays an essential functional role for casein calcium binding and micelles formation, some of such existing approaches for recombinant expression of dairy proteins are clearly suboptimal. Furthermore, when dairy proteins are expressed in yeast, phosphorylation produces an energetic toll since it consumes ATP molecules, that is translated to low productivity.

In addition, existing approaches to recombinant expression of dairy proteins in heterologous systems can lead to an altered, and potentially undesirable, isoelectic point in the mature expressed protein due to lack of phosphorylation groups on serine moieties. Amino acid phosphorylation adds negatively charged groups to the protein which reduces the pl of the protein. For example native beta-casein pl is 4.62 while non-phosphorylated beta-casein pl is above 5. This in turn provides problems for some industrial applications of the expressed proteins such as those which rely on the protein precipitation, coagulation and enzymatic activity at a precise pH (Okigbo, L. M., Richardson, G. H., Brown, R. J. and Ernstrom, C. A., 1985. Interactions of calcium, pH, temperature, and chymosin during milk coagulation. Journal of Dairy Science, 68 (12), pp. 3135-3142.).

It is therefore an object of the invention to provide an alternative approach to producing recombinant milk or dairy proteins that alleviate one or more of the problems found with existing approaches, and/or at least provide the public with a useful choice.

### SUMMARY OF THE INVENTION

The invention provides a novel approach to expressing dairy proteins in heterologous systems. This approach involves modifying the dairy proteins by replacement of potentially phosphorylated amino acids in the dairy proteins, with negatively charged amino acids. The modified dairy proteins including the negatively charges amino acids mimic phosphorylated dairy proteins thereby promoting micelle formation and associated benefits. In addition, replacement of the potentially phosphorylated amino acids in the dairy proteins, with negatively charged amino acids allows for targeted manipulation of the pl of the mature expressed dairy proteins, thereby providing recombinantly expressed dairy proteins with improved properties relative to the recombinantly expressed dairy proteins of the prior art. Furthermore, the negative charge facilitates Ca²⁺ ion-binding which is important for the nutritional value as well as the protein functionality.

### Modified Dairy Protein

In the first aspect the invention provides a modified dairy protein in which at least one potentially phosphorylated amino acid has been replaced with a negatively charged amino acid.

### Target Phosphorylation Site

In one embodiment the potentially phosphorylated amino acid is selected from serine(S) and threonine (T). In one embodiment the potentially phosphorylated amino acid is serine(S). In a further embodiment the potentially phosphorylated amino acid is threonine (T).

In one embodiment the potentially phosphorylated amino acid has been experimentally shown to be phosphorylated.

In a further embodiment the potentially phosphorylated amino acid is has been predicted to be phosphorylated. In one embodiment the prediction was made using suitable sequence analysis software.

### Negatively Charged Amino Acid

In one embodiment the negatively charged amino acid is selected from aspartate (D) and glutamate (E). In one embodiment the negatively charged amino acid is aspartate (D). In a further embodiment the negatively charged amino acid is glutamate (E).

### Properties of the Modified Dairy Protein

### Calcium Binding

In one embodiment the modified dairy protein, when recombinantly expressed in a heterologous expression system, is capable of binding calcium.

In a further embodiment the modified dairy protein has an improved capacity to binding calcium relative to the unmodified dairy protein when it is recombinantly expressed in the same heterologous expression system.

### Capability to Form Micelles

In one embodiment the modified dairy protein, when recombinantly expressed in a heterologous expression system, is capable forming a micelle.

In a further embodiment the modified dairy protein has an improved capacity to form a micelle, relative to the unmodified dairy protein when it is recombinantly expressed in the same heterologous expression system.

### Modified pl

In one embodiment the modified dairy protein, when recombinantly expressed in a heterologous expression system, has an altered pl relative to that of the unmodified dairy protein when it is recombinantly expressed in the same heterologous expression system.

In one embodiment the modified dairy protein, when recombinantly expressed in a heterologous expression system, has an increased pl relative to that of the unmodified dairy protein when it is recombinantly expressed in the same heterologous expression system.

In one embodiment the modified dairy protein, when recombinantly expressed in a heterologous expression system, has a decreased pl relative to that of the unmodified dairy protein when it is recombinantly expressed in the same heterologous expression system.

In some embodiments, the modified dairy protein has an isoelectric point (pl) identical or similar to the unmodified dairy protein.

In one embodiment the pl of the modified dairy protein is within +/−1.0, +/−0.9, preferably +/−0.8, +/−0.7, preferably +/−0.6, preferably +/−0.5, preferably +/−0.4, preferably +/−0.3, preferably +/−0.2, preferably 0.1 units relative to the unmodified dairy protein.

### Thermal Stability

In one embodiment the modified dairy protein, when recombinantly expressed in a heterologous expression system, has an altered thermal stability relative to that of the unmodified dairy protein when it is recombinantly expressed in the same heterologous expression system.

In one embodiment the modified dairy protein, when recombinantly expressed in a heterologous expression system, has an increased thermal stability relative to that of the unmodified dairy protein when it is recombinantly expressed in the same heterologous expression system.

In one embodiment the modified dairy protein, when recombinantly expressed in a heterologous expression system, has a decreased thermal stability relative to that of the unmodified dairy protein when it is recombinantly expressed in the same heterologous expression system.

### Zeta Potential

In one embodiment the modified dairy protein, when recombinantly expressed in a heterologous expression system, has an altered zeta potential relative to that of the unmodified dairy protein when it is recombinantly expressed in the same heterologous expression system.

In one embodiment the modified dairy protein, when recombinantly expressed in a heterologous expression system, has an increased zeta potential relative to that of the unmodified dairy protein when it is recombinantly expressed in the same heterologous expression system.

In a preferred embodiment the modified dairy protein, when recombinantly expressed in a heterologous expression system, has a decreased zeta potential relative to that of the unmodified dairy protein when it is recombinantly expressed in the same heterologous expression system.

### Dairy Proteins

In one embodiment, the unmodified dairy protein is a casein protein.

In one embodiment the casein protein is selected from αs1-casein, αs2-casein, β-casein, and κ-casein. In one embodiment the casein protein is bovine A2 β-casein. In one embodiment the bovine A2 β-casein has proline (P) at position 67 in its amino-acid sequence.

### Similarity to Known Dairy Proteins

In one embodiment the modified dairy protein has at least 90%, preferably at least 91%, preferably at least 92%, preferably at least 93%, preferably at least 94%, preferably at least 95%, preferably at least 96%, preferably at least 97%, preferably at least 98%, preferably at least 98% amino acid sequence identity to a known dairy protein of the type described above.

In one embodiment apart from the modifications made in producing the modified dairy protein, the modified dairy protein is otherwise the same in sequence as the unmodified dairy protein.

In an alternative embodiment the modified dairy protein included further modifications relative to the unmodified dairy protein.

### Polynucleotide Encoding the Modified Dairy Protein

In a further aspect the invention provides a polynucleotide encoding the modified dairy protein of the invention.

### Construct

In a further aspect the invention provides a construct comprising the polynucleotide encoding the modified dairy protein of the invention.

In one embodiment the construct comprises:
a) a promoter sequence
b) a polynucleotide encoding a plant-derived dairy protein, or dairy protein analogue of the invention, and
c) a terminator sequence

In one embodiment the construct includes a sequence encoding a signal, or transit peptide, to target the plant-derived dairy protein to a desired sub-cellular compartment.

In one embodiment the promoter is a seed preferred promoter.

### Host Cell

In a further aspect the invention provides a host cell comprising a polynucleotide or construct of the invention.

Preferably the host cell is heterologous to the species from which the unmodified dairy protein is derived.

In a preferred embodiment the host cell is a plant cell.

### Host Organism

In a further aspect the invention provides a host organism comprising a polynucleotide or construct of the invention.

Preferably the organism is heterologous to the species from which the unmodified dairy protein is derived.

In a preferred embodiment the organism is a plant.

### Plant Part, Tissue, Propagule or Progeny

In a further aspect the invention provides a plant part, tissue, propagule or progeny plant, comprising a polynucleotide or construct of the invention.

In a preferred embodiment the plant part is a seed.

In a preferred embodiment the plant tissue is seed tissue.

### Method of Producing a Modified Dairy Protein

In further aspect the invention provides for producing a modified dairy protein of the invention, the method comprising expressing a polynucleotide encoding the modified dairy protein, or the modified dairy protein, of the invention in a host cell or organism.

In one embodiment the method comprises the step of transforming the host cell or organism the polynucleotide.

Preferably the host cell is heterologous to the species from which the unmodified dairy protein is derived.

In a preferred embodiment the host cell is a plant cell.

Preferably the organism is heterologous to the species from which the unmodified dairy protein is derived.

In a preferred embodiment the organism is a plant.

In a preferred embodiment the modified dairy protein is expressed in a seed of the plant.

In a further embodiment the modified dairy protein is expressed in a seed tissue of the plant.

In one embodiment the transformation is transient. In a further embodiment the transformation is stable.

In a further embodiment the method comprises the step of modifying the polynucleotide to encode the modified dairy protein.

In a further embodiment the method comprises the step of testing the expressed modified dairy protein with respect to at least one of its:
a) capacity to bind calcium,
b) capacity to form a micelle,
c) pl,
d) thermal stability, and
e) zeta potential

In a further embodiment the modified dairy protein is selected based on at least one of a)-e).

In a further embodiment the method comprises the step of purifying the modified dairy protein from the host cell or organism.

In a further embodiment the invention provides a modified dairy protein produced by a method of the invention.

Product comprising a modified dairy protein of the invention In a further aspect of the invention provides a food or beverage product or ingredient comprising one or more modified dairy protein of the invention.

Preferably, the food or beverage product or ingredient is selected from milk, cream, chocolate, butter, cheese, fermented yoghurt, ice-cream, infant formula, protein beverages, custards, buttermilk, milk powders, margarine, whey-protein concentrates and isolates, milk protein concentrates or isolates and hydrolysed milk proteins.

### Use of the Modified Dairy Protein of the Invention.

In a further aspect of the invention provides use of the modified dairy protein of the invention in preparation of a food or beverage product or ingredient.

### Method for Producing a Food or Beverage Product or Ingredient

In a further aspect of the invention provides a method for producing a food or beverage product or ingredient, the method including the step of processing or incorporating the modified dairy protein of the invention, or produced by the method of the invention, into food or beverage product or ingredient.

Preferably, the food or beverage product or ingredient is selected from milk, cream, chocolate, butter, cheese, fermented yoghurt, ice-cream, infant formula, protein beverages, custards, buttermilk, milk powders, margarine, whey-protein concentrates and isolates, milk protein concentrates or isolates and hydrolysed milk proteins.

### Dairy Product Substitute

In a preferred embodiment the food or beverage product is a dairy product substitute.

In one embodiment the dairy product substitute is selected from a milk substitute, a cream substitute, a whipping cream substitute, a mayonnaise substitute, an ice cream substitute, a cheese substitute, and a yoghurt substitute.

In one embodiment the dairy product substitute comprises at least one additional component selected from at least one emulsifier, at least one stabilizer, at least one flavor compound, at least one colour compound, at least one preservative, at least one sweetening compound, at least one nutritional compound (i.e. vitamin, mineral, antioxidant, bioactive or other source of additional nutritional benefit).

### DETAILED DESCRIPTION OF THE INVENTION

The invention relates to modified dairy proteins that can retain beneficial properties such as calcium binding capacity, capacity to form micelles, the desired isoelectic point (pl), thermal stability and zeta potential, when expressed in heterologous host cells and organisms.

The modified dairy proteins can be produced without the use of animals, and find a wide variety of uses, particularly in the food and beverage industry.

### Dairy Proteins

The terms “dairy protein/s” and “milk protein/s” can be used interchangeably.

Milk contains two main groups of proteins, namely caseins and whey proteins. There are four types of caseins, denoted as αs1-casein, αs2-casein, β-casein and κ-caseins which represent approximately 37, 10, 35 and 12% of the whole casein respectively. Each of the four caseins exhibits variability in the degree of phosphorylation and glycosylation. All caseins are phosphorylated: most of the αs1-casein molecules contain 8 PO₄ residues but some contain 9; αs2-casein contains 10, 11, 12 or 13 mol PO₄/mol; β-casein usually contains 5 mol PO₄/mol but occasionally 4 mol PO₄/mol; K-casein contains 1 mol PO₄/mol. κ-casein is the only casein which is normally glycosylated and contains galactose, galactosamine and N-acetyl neuraminic acid. A further heterogeneity in caseins arises from the occurrence of genetic polymorphism, which is due to either substitutions or, rarely, deletions of amino acids in the caseins because of mutations causing changes in base sequences in the genes.

In comparison to typical globular proteins, the structures of caseins are quite unique. An interesting feature of all caseins is the amphiphilicity of the primary structures. All caseins have regions that are acidic, basic or neutral and hydrophilic or hydrophobic. The caseins, compared to typical globular proteins which have mainly α-helical and β-sheet structures, contain less secondary structures. All major caseins also interact with each other to form various complexes of different sizes and shapes. Caseins bind calcium, and the extent of binding is directly related to the number of phospho-serine residues in the molecule.

Milk proteins, especially casein proteins, are used in a wide variety of functional and nutritional applications and have a range of properties that make them particularly suitable in the formation of and incorporation within food-grade materials. They are effective encapsulating materials, possess film-forming and emulsifying properties that allow them to act as stabilisers in emulsion-based systems, act as carriers of other materials and may be easily formed into a dried state.

As ingredients in food products, milk proteins introduce a range of sensory characteristics to the food products and the person ingesting them, including inducing satiety, improved mouth-feel, viscosity and structure, flavour and they act as substrates for other flavours and aromatic compounds in multi-ingredient compositions.

The methods described herein seek to replicate some or all of these characteristics in a modified milk protein, removing the need for, or providing an alternative to animal-based milk protein production.

TABLE 1
Examples of milk or dairy proteins known in the art

| Milk protein | Source | Type | Accession No | SEQ ID NO |
|---|---|---|---|---|
| αs1- casein | Bovine | Protein | P02662 | 6 |
| αs1- casein | Bovine | DNA (cds) | X00564 | 7 |
| αs2- casein | Bovine | Protein | P02663 | 8 |
| αs2- casein | Bovine | DNA (cds) | M16644 | 9 |
| A1β-casein | Bovine | Protein | P02666 | 10 |
| A1 β-casein | Bovine | DNA (cds) | X06359 | 11 |
| A2 β-casein | Bovine | Protein | P02666 | 1 |
| A2 β-casein | Bovine | DNA (cds) | M15132 | 5 |
| κ-casein | Bovine | Protein | P02668 | 12 |
| κ-casein | Bovine | DNA (cds) | X00565 | 13 |
| αs1- casein | Goat | Protein | P18626 | 14 |
| αs1- casein | Goat | DNA (cds) | X59836 | 15 |
| αs2- casein | Goat | Protein | P33049 | 16 |
| αs2- casein | Goat | DNA (cds) | X65160 | 17 |
| A2 β-casein | Goat | Protein | P33048 | 18 |
| A2 β-casein | Goat | DNA (cds) | AH001195 | 19 |
| κ-casein | Goat | Protein | P02670 | 20 |
| κ-casein | Goat | DNA (cds) | D14373 | 21 |
| αs1- casein | Sheep | Protein | P04653 | 22 |
| αs1- casein | Sheep | DNA (cds) | X03237 | 23 |
| αs2- casein | Sheep | Protein | P04654 | 24 |
| αs2- casein | Sheep | DNA (cds) | X03238 | 25 |
| β-casein | Sheep | Protein | P11839 | 26 |
| β-casein | Sheep | DNA (cds) | X16482 | 27 |
| κ-casein | Sheep | Protein | P02669 | 28 |
| κ-casein | Sheep | DNA (cds) | X51822 | 29 |
| αs1- casein | Camel | Protein | O97943 | 30 |
| αs1- casein | Camel | DNA (cds) | AJ012628 | 31 |
| αs2- casein | Camel | Protein | O97944 | 32 |
| αs2- casein | Camel | DNA (cds) | AJ012629 | 33 |
| β-casein | Camel | Protein | Q9TVD0 | 34 |
| β-casein | Camel | DNA (cds) | AJ012630 | 35 |
| κ-casein | Camel | Protein | P79139 | 36 |
| κ-casein | Camel | DNA (cds) | Y10082 | 37 |

### Source of Unmodified Dairy Proteins

The unmodified dairy or milk proteins for use in the invention may be from any suitable source.

In one embodiment the unmodified dairy or milk proteins for use in the invention may be from a source selected from sheep, deer, camel, goat, human and bovine organisms.

In a preferred embodiment the unmodified dairy or milk proteins for use in the invention are from bovine organisms.

### Phopshorylation of Dairy Proteins

In native dairy proteins, phosphorylation of caseins is an important post-translational modification occurring after the synthesis of the polypeptide chains under the action of protein kinases. Bovine kinases phosphorylate Ser or Thr sites by recognizing the tripeptide sequence Ser/Thr-X-Glu/SerP/Asp, where X represents any amino acid residue and P indicates phosphorylation (Fang et al., J. Dairy Sci. 99:8168-8177). This post-translational modification allows caseins to interact with and bind to calcium phosphate to form casein micelles.

The bovine kinases involved in phosphorylation are absent in plants and other non-bovine organisms such as E. coli (Simons, G., van den Heuvel, W., Reynen, T., Frijters, A., Rutten, G., Slangen, C. J., Groenen, M., de Vos, W. M. and Siezen, R. J., 1993. Overproduction of bovine β-casein in Escherichia coli and engineering of its main chymosin cleavage site. Protein Engineering, Design and Selection, 6 (7), pp. 763-770.). Moreover, the phosphorylation process by kinases requires ATP thus energetically inefficient and can impact the modified organism growth.

The present invention addresses this issue by replacing Ser and/or Thr with either Asp or Glu. Both Asp and Glu are negatively charged side chains above pH 3.5, and provide binding sites for positively charged calcium ions. See FIG. 2.

### Phosphorylation Sites

The present invention involves replacing potentially phosphorylated amino acids with negatively charged amino acids.

Some phosphorylated amino acids in dairy proteins have been experimentally verified. These include for example Ser15, Ser17, Ser18, Ser19, and Ser35 in Bovine beta-Casein (Huppertz et al., 2018. The caseins: Structure, stability, and functionality. In Proteins in food processing pp. 49-92), as indicated in FIG. 1. Further phosphorylation sites can be verified in other dairy proteins in the same way. Alpha-S1-casein is phosphorylated at Ser41, Ser45, Ser47, Ser64, Ser66, Ser67, Ser68, Ser75, and Ser115. Alpha-S2-casein is phosphorylated at Ser8, Ser9, Ser10, Ser16, Ser56, Ser57, Ser58, Ser61, Ser129 and Ser131 (Huppertz, T., Fox, P. F. and Kelly, A. L., 2018. The caseins: Structure, stability, and functionality. In Proteins in food processing pp. 49-92). Other phosphorylation versions exist based on different polymorphism within caseins.

Potentially phopsphorylated amino acids also include the Ser and Thr residues in the consensus tripeptide sequence Ser/Thr-X-Glu which can be readily identified in dairy protein sequences by those skilled in the art.

Other potential phosphorylation sites can be identified using in-silico phosphorylation prediction algorithms such as the NetPhos algorithm http://www.cbs.dtu.dk/services/NetPhos/(Blom et al., 1999; Sequence and structure-based prediction of eukaryotic protein phosphorylation sites. Journal of molecular biology, 294 (5), pp. 1351-1362.). This is exemplified for Bovine beta-Casein in the present Examples, and illustrated in FIG. 1.

Number of Phosphorylation Sites Replaced with Negatively Charged Amino Acids.

In one embodiment at least 1, preferably at least 2, preferably at least 3, preferably at least 4, preferably at least 5, preferably at least 6, preferably at least 7, preferably at least 8, preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20, phosphorylation sites, or potential phosphorylation site, is/are replaced with a negatively charged amino acid/s.

In a further embodiment no more than 20, preferably more than 19, preferably more than 18, preferably more than 17, preferably more than 16, preferably more than 15, preferably more than 14, preferably more than 13, preferably more than 12, preferably more than 11, preferably more than 10, preferably more than 9, preferably more than 8, preferably more than 7, preferably more than 6, preferably more than 5, preferably more than 4, preferably more than 3, preferably more than 2, preferably more than 1, phosphorylation site/s, or potential phosphorylation site/s, is/are replaced with a negatively charged amino acid/s.

### Similarity of the Modified Dairy Protein to Known Unmodified Dairy Proteins

In one embodiment the modified dairy proteins of the invention have at least 90% preferably at least 91%, preferably at least 92%, preferably at least 93%, preferably at least 94%, preferably at least 95%, preferably at least 96%, preferably at least 97%, preferably at least 98%, preferably at least 99% amino acid sequence identity to a known dairy protein, including but not limited to those listed in Table 1 above.

Polypeptide sequence identity can be determined in the following manner. The subject polypeptide sequence is compared to a candidate polypeptide sequence using BLASTP (from the BLAST suite of programs, version 2.2.5 [November 2002]) in bl2seq, which is publicly available from the NCBI website on the World Wide Web at ftp://ftp.ncbi.nih.gov/blast/.

The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

A preferred method for calculating polypeptide % sequence identity is based on aligning sequences to be compared using Clustal X (Jeanmougin et al., 1998, Trends Biochem. Sci. 23, 403-5.)

### Properties of the Modified Dairy Proteins

### Calcium Binding

In one embodiment the modified dairy protein, when recombinantly expressed in a heterologous expression system, is capable of binding calcium.

The calcium binding capacity can be measured flame photometry for example.

The capability of the modified dairy protein of the invention to bind calcium can be assessed by methods well known to those skilled in the art such as, for example, flame photometry, ICP-AES, atomic absorption spectroscopy, isothermal titration calorimetry technique (e.g. Luo et al 2020. Deciphering calcium-binding behaviors of casein phosphopeptides by experimental approaches and molecular simulation. Food & Function, 11 (6), pp. 5284-5292; Canabady-Rochelle, L. S. and Mellema, M., 2010. Physical-chemical comparison of cow's milk proteins versus soy proteins in their calcium-binding capacities. Colloids and Surfaces A: Physicochemical and Engineering Aspects, 366 (1-3), pp.110-112)

In some embodiments modified dairy protein has at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 105%, preferably at least 110%, preferably at least 115%, preferably at least 120%, preferably at least 125% of the calcium binding capacity of the unmodified dairy protein.

In a further embodiments the modified dairy protein has at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 130%, preferably at least 135%, preferably at least 140%, preferably at least 145%, preferably at least 150%, preferably at least 1155%, preferably at least 160%, preferably at least 165%, preferably at least 170%, preferably at least 175%, preferably at least 180% preferably at least 185%, preferably at least 190%, preferably at least 195%, preferably at least 200% of the calcium binding capacity of the unmodified dairy protein, when the modified dairy protein and the unmodified dairy protein are expressed in the same heterologous expression system.

In a preferred embodiment, the heterologous expression system is a plant expression system.

### Enhanced Capability to Form Micelles

In one embodiment the modified dairy protein, when recombinantly expressed in a heterologous expression system, is capable forming a micelle.

The capability of the modified dairy protein of the invention to form micelles can be assessed by methods well known to those skilled in the art such as dynamic light scattering, microscopy methods (scanning electron microscopy) (e.g. Chu, B., Zhou, Z., Wu, G. and Farrell Jr, H. M., 1995. Laser light scattering of model casein solutions: effects of high temperature. Journal of Colloid and Interface Science, 170 (1), pp. 102-112.; Dalgleish, D. G., Spagnuolo, P. A. and Goff, H. D., 2004. A possible structure of the casein micelle based on high-resolution field-emission scanning electron microscopy. International Dairy Journal, 14 (12), pp. 1025-1031.)

In some embodiments modified dairy protein has at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 105%, preferably at least 110%, preferably at least 115%, preferably at least 120%, preferably at least 125% of the micelle forming capacity of the unmodified dairy protein.

In a further embodiments the modified dairy protein has at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 130%, preferably at least 135%, preferably at least 140%, preferably at least 145%, preferably at least 150%, preferably at least 1155%, preferably at least 160%, preferably at least 165%, preferably at least 170%, preferably at least 175%, preferably at least 180% preferably at least 185%, preferably at least 190%, preferably at least 195%, preferably at least 200% of the micelle forming capacity of the unmodified dairy protein, when the modified dairy protein and the unmodified dairy protein are expressed in the same heterologous expression system.

In a preferred embodiment, the heterologous expression system is a plant cell- or plant-based expression system.

### Modifed pl

In one embodiment the modified dairy protein, when recombinantly expressed in a heterologous expression system, has an altered pl relative to that of the unmodified dairy protein when it is recombinantly expressed in the same heterologous expression system.

Preferably, the modified dairy protein has an isoelectric point (pl) identical or similar to the unmodified dairy protein.

In one embodiment the pl of the modified dairy protein is within +/−1.0, preferably +/−0.9, preferably +/−0.8, +/−0.7, preferably +/−0.6, preferably +/−0.5, preferably +/−0.4, preferably +/−0.3, preferably +/−0.2, preferably 0.1 units relative to the unmodified dairy protein.

In one embodiment the modified and unmodified dairy protein are expressed in the same heterologous expression system.

In a preferred embodiment, the heterologous expression system is a plant cell- or plant-based expression system.

The pl of the modified dairy protein of the invention can be assessed by methods well known to those skilled in the art (e.g. Righetti, P. G., 2000. Isoelectric focusing: theory, methodology and application. Elsevier; Righetti, P. G., 2004. Determination of the isoelectric point of proteins by capillary isoelectric focusing. Journal of chromatography A, 1037 (1-2), pp. 491-499.)

### Methods for Producing Polynucleotides of the Invention

Methods for producing polynucleotides (that can be used to express the proteins and analogues of the invention) are well known to those skilled in the art, and include use of cloning and recombinant DNA technologies. These technologies may involve modification of an existing polynucleotide encoding a dairy protein. Alternatively the polynucleotide can be synthesised in its entirety by methods commonly used by those skilled in the art, and available commercially as a service from numerous well-known providers (e.g. GeneArt, Thermo Fisher Scientific).

The polynucleotides of the invention, encoding the modified dairy proteins of the invention, may be codon optimised to resemble the codon usage of the cell or organism in which the modified dairy proteins are expressed. Codons may be optimised using known tools (such as www.genewiz.com). This may result in improved gene expression and increased the translational efficiency, by accommodating the codon bias of the host.

### Methods for Producing Constructs and Vectors

The genetic constructs of the present invention comprise one or more polynucleotide sequences of the invention and/or polynucleotides encoding polypeptides of the invention, and may be useful for transforming, for example, bacterial, fungal, insect, mammalian or plant organisms. The genetic constructs of the invention are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

### Methods for Producing Host Cells Comprising Polynucleotides, Constructs or Vectors

The invention provides a host cell which comprises a genetic construct or vector of the invention.

Host cells comprising genetic constructs, such as expression constructs, of the invention are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides of the invention. Such methods may involve the culture of host cells in an appropriate medium in conditions suitable for or conducive to expression of a polypeptide of the invention. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

### Methods for Producing Plant Cells and Plants Comprising Constructs and Vectors

The invention further provides plant cells which comprise a genetic construct of the invention, and plant cells modified to alter expression of a polynucleotide or polypeptide of the invention, or used in the methods of the invention. Plants comprising such cells also form an aspect of the invention.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

### Methods for Genetic Manipulation of Plants

A number of plant transformation strategies are available (e.g. Birch, 1997, Ann Rev Plant Phys Plant Mol Biol, 48, 297; Hellens et al., 2000, Plant Mol Biol 42:819-32; Hellens et al., Plant Meth 1:13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Genetic constructs for expression of genes in transgenic plants typically include promoters for driving the expression of one or more cloned polynucleotide, terminators and selectable marker sequences to detect presence of the genetic construct in the transformed plant.

The promoters suitable for use in the constructs of this invention are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues respond to internal developmental signals or external abiotic or biotic stresses are described in the scientific literature. Exemplary promoters are described, e.g., in WO 02/00894 and WO2011/053169, which is herein incorporated by reference.

Preferred regulatory sequences (promoters, signal/transit peptides and terminators) include those associated with seed storage proteins. Suitable seed storage proteins include but are not limited to glycenin (soybean), vicilin, convicilin, legumin (pea), cruciferin, napin (canola), patatin (potato).

In some embodiments the promoters used are seed preferred promoters. In one embodiment the seed preferred promoter is selected from those in the Table 2 below.

TABLE 2
Seed preferred promoters

| Source | Promoter | SEQ ID NO: |
|---|---|---|
| Brassica napus | Cruciferin A | 47 |
| Phaseolus vulgaris | Phaseolin | 48 |
| Glycine max | Glycinin G1 | 49 |
| Glycine max | Glycinin G4 | 50 |
| Glycine max | Glycinin G2 | 51 |
| Glycine max | Beta-conglycinin α-subunit promoter | 52 |

The expression constructs of the invention also preferably include a signal or transit peptide fused to the N-terminus of the plant derived dairy protein or analogue to direct it to a storage organelle (storage vacuole, ER, apoplast, etc.). A C-terminal retention signal may also be include when targeting some of the storage organelles.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9: 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci.104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); cereals (U.S. Pat. No. 6,074,877); pear (Matsuda et al., 2005, Plant Cell Rep. 24 (1): 45-51); Prunus (Ramesh et al., 2006 Plant Cell Rep. 25 (8): 821-8; Song and Sink 2005 Plant Cell Rep. 2006; 25 (2): 117-23; Gonzalez Padilla et al., 2003 Plant Cell Rep.22 (1): 38-45); strawberry (Oosumi et al., 2006 Planta. 223 (6): 1219-30; Folta et al., 2006 Planta Apr 14; PMID: 16614818), rose (Li et al., 2003), Rubus (Graham et al., 1995 Methods Mol Biol. 1995; 44:129-33), tomato (Dan et al., 2006, Plant Cell Reports V25: 432-441), apple (Yao et al., 1995, Plant Cell Rep. 14, 407-412), Canola (Brassica napus L.). (Cardoza and Stewart, 2006 Methods Mol Biol. 343:257-66), safflower (Orlikowska et al, 1995, Plant Cell Tissue and Organ Culture 40:85-91), ryegrass (Altpeter et al., 2004 Developments in Plant Breeding 11 (7): 255-250), rice (Christou et al., 1991 Nature Biotech. 9:957-962), maize (Wang et al., 2009 In: Handbook of Maize pp. 609-639) and Actinidia eriantha (Wang et al., 2006, Plant Cell Rep. 25,5:425-31). Transformation of other species is also contemplated by the invention. Suitable methods and protocols are available in the in scientific literature.

The polynucleotides of the invention, and their encoded plant-derived dairy proteins and analogues, can also be conveniently expressed and test via transient expression in leaves of transgenic plants, such as Nicotian benthamiana, using Agrobacterium infiltration (Kapila, J., De Rycke, R., Van Montagu, M. and Angenon, G. (1997) An Agrobacterium-mediated transient gene expression system for intact leaves. Plant Sci. 122, 101-108).

### Plant Cells and Plants

The plant cells and plants in which the modified dairy proteins are expressed, may be from any plant species.

In one embodiment the plant cell or plant, is from a gymnosperm plant species.

In a further embodiment the plant cell, or plant, is from an angiosperm plant species.

In a further embodiment the plant cell, or plant, is from a from dicotyledonous plant species.

In a further embodiment the plant cell, or plant, is from a monocotyledonous plant species.

In one embodiment the plant or plant cell is selected from, but not limited to: soybean, tobacco, pea, chickpea, bean, lupin, fava bean, lentils or any other legume, tapioca, rice, barley, wheat, oats, maize, tomato, potato, canola, oil seed rape, sunflower, safflower, coconut, almond and hemp.

### Purification of Modified Dairy Proteins of the Invention

After transient or stable transformation of plant cells or plants, the expressed modified dairy proteins of the invention may be purified using one or more techniques such as, but not limited to micellization techniques or alkaline extraction/isoelectric precipitation. The isolation technique used will be selected depending on the suitability of each technique to the plant protein source and how the extraction techniques may influence the functional properties of the proteins produced. Considerations of solubility, pH, water binding capacity, temperature and structural integrity should be taken into account, together with the final desired use of the modified dairy protein when deciding on the protein isolation technique to be used.

Following protein extraction and isolation, proteins may be purified using selective precipitation methods, centrifugation and/or filtration.

### Use of the Modified Dairy Proteins of the Invention in Food and Beverages

The subsequent isolated modified dairy proteins may be used as ingredients in food and beverage products, incorporating one or many of the functional characteristics found in native milk and dairy proteins. Such products may include but are not limited to milk, butter, buttermilk, cheese, cream, ice-cream, whey, casein, yoghurt, milk powders, infant formulas or pet and animal dairy products.

The entire disclosures of all applications, patents and publications cited above and below, if any, are herein incorporated by reference.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner. In some embodiments, the term “comprising” (and related terms such as “comprise and “comprises”) can be replaced by “consisting of” (and related terms “consist” and “consists”).

### BRIEF DESCRIPTION OF THE DRAWINGS

FIG. 1 shows the sequence of the dairy protein Bovine beta-Casein (A2). Experimentally verified phosphoserines are highlighted in bold font. Other potential phosphorylation sites identified by the applicant's analysis are highlighted with underlining.

FIG. 2 shows a comparison of between phosphoserine (serine is typically phosphorylated in bovine dairy proteins as discussed herein), aspartic acid and glutamic acid).

FIG. 3 shows the schematically represented structure of constructs used for expressing the modified dairy proteins of the invention in plants. Further detail is provided in Example 2.

FIG. 4(A) shows representative leaf samples transformed with beta-caseins fused to the vacuolar signal peptide; Lane 1-Positive control (beta-casein 0.5 μg), Lane 2-4: WT; Lane M: protein ladder; Lane 5-9: extract from leaves transformed with constructs 1, 3, 5 and 7 respectively. Bands corresponding to beta caseins are marked with an arrow.

FIG. 4(B) shows representative leaf samples transformed with beta-caseins fused to the apoplast signal peptide with ER retention site at the C-terminal end of the proteins; Lane 1-Positive control (beta-casein 0.5 μg), Lane 2-3: extract from leaves transformed with constructs 2 and 4; Lane M: protein ladder; Lane 4-6: WT; Lane 7: extract from leaves transformed with construct 6; Lane 8-9: extracts from leaves transformed with construct 8. Bands corresponding to beta caseins are marked with an arrow.

FIG. 5 shows SDS-PAGE (Coomassie stain) analysis of β-casein protein designs (BC1, BC2, BC3, BC4 and BC5) and the positive control (Sigma β-casein-1 μg). Black arrow-expected size of beta-casein. White arrow-casein degradation products.

FIG. 6: shows CD [mDeg] as a function of wavelength for the far-UV CD of different β-casein at 5, 15, 25, 35, 45, 55, 65, 75, 85, 95° C. A. positive control-beta-casein protein obtained from Sigma (C6905); B. Native beta casein expressed in E. Coli (BC1); C. modified beta-casein expressed in E. Coli (BC2); D. modified beta-casein expressed in E. Coli (BC3). E. modified beta-casein expressed in E. Coli (BC4). F. modified beta-casein expressed in E. Coli (BC5).

FIG. 7 shows CD [mDeg] as a function of wavelength for the far-UV CD of different β-casein at 5° C., 95° C., and 5° C. after incubation at 95° C. A. positive control—beta-casein protein obtained from Sigma (C6905); B. Native beta casein expressed in E. Coli (BC1); C. modified beta-casein expressed in E. Coli (BC2); D. modified beta-casein expressed in E. Coli (BC3). E. modified beta-casein expressed in E. Coli (BC4). F. modified beta-casein expressed in E. Coli (BC5).

FIG. 8 shows total protein in 100 μl as a function of pH measured for the β-casein analogues. The fitting is shown as gray and black lines for the supernatant and pellets, respectively. A. positive control-beta-casein protein obtained from Sigma (C6905); B. Native beta casein expressed in E. Coli (BC1); C. modified beta-casein expressed in E. Coli (BC2); D. modified beta-casein expressed in E. Coli (BC3). E. modified beta-casein expressed in E. Coli (BC4). F. modified beta-casein expressed in E. Coli (BC5).

### EXAMPLES

### Example 1. Design of Modified Proteins of the Invention

### Introduction

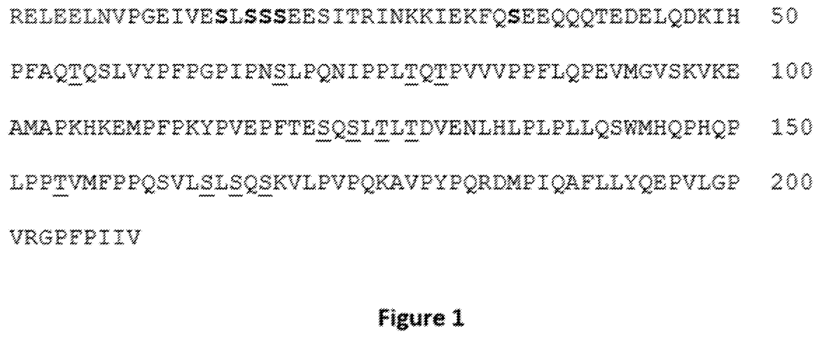

The sequence of the dairy protein Bovine beta-Casein (A2) is shown in FIG. 1.

Bovine beta-Casein has experimentally verified phosphorylation sites on Ser15, Ser17, Ser18, Ser19, and Ser35 (as indicated in bold font in FIG. 1). Of these, the first four form a center of phosphorylation (Huppertz et al., 2018; The caseins: Structure, stability, and functionality. In Proteins in food processing pp. 49-92).

The negative charges obtained from the phosphoserines have an essential functional role for casein micelles formation. The caseins are held together by a combination of hydrophobic interactions between protein molecules and electrostatic interactions between phosphoserine-rich regions of the α- and β-caseins and calcium creating calcium phosphate bonds. Moreover, the phosphoserine-rich residues play a fundamental role in providing the correct functionality to caseins, as intrinsically related to the emulsion stabilization by β-caseins.

However, casein phosphorylation does not occur in transgenic plants (and other non-bovine systems) expressing recombinant beta-casein (Philip et al., 2001, Processing and localization of bovine β-casein expressed in transgenic soybean seeds under control of a soybean lectin expression cassette. Plant Science, 161 (2), pp. 323-335).

The applicant's invention involves replacing phosphorylated amino acids with negatively charged amino acids using protein engineering methods

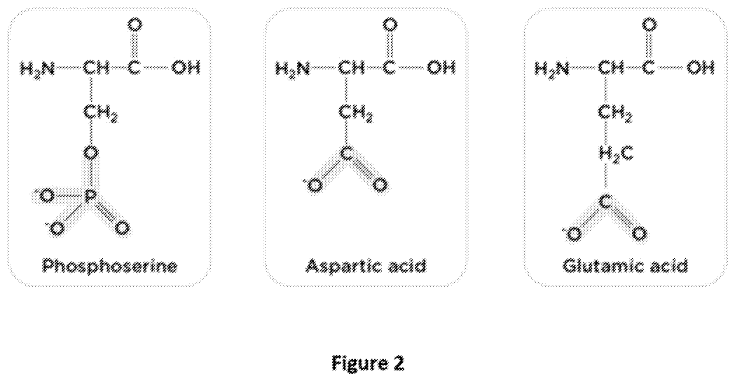

FIG. 2 shows a comparison of between phosphoserine (serine is typically phosphorylated in bovine dairy proteins as discussed above), aspartic acid and glutamic acid).

The negatively charged amino acids aspartic acid and glutamic acid have capacity to bind positively charged calcium. Such calcium binding is a key requirement of dairy protein function.

The invention therefore provides modified dairy proteins which when expressed in heterologous expression systems, include negatively charged amino acids in place of phosphorylated amino acids, providing recombinantly expressed modified dairy proteins that replicate the functionality of replicate the functionality of endogenously expressed dairy proteins.

To reveal more potential sites of phosphorylation, in addition to the experimentally known phosphoserine sites, in-silico phosphorylation prediction was performed using the NetPhos algorithm http:/www.cbs.dtu.dk/services/NetPhos/(Blom, N., Gammeltoft, S. and Brunak, S., 1999. Sequence and structure-based prediction of eukaryotic protein phosphorylation sites. Journal of molecular biology, 294 (5), pp. 1351-1362). The applicant applied this approach to the beta Casein (A2)—mature peptide (UniProt accession no. P02666-SEQ ID NO: 1).

Further potential phosphorylation sited were identified, as indicated with underlined font in FIG. 1.

### Modified Dairy Protein Design

Several different modified beta-casein protein sequences were designed, in which one or more phosphorylation sites, or potential phosphorylation sites, were replaced with negatively charges amino acids, to address essential functions valued by the food and beverage industry, and specifically to:
Mimic phosphorylation to various levels to allow different calcium-binding and micelle formation properties, while maintaining the native isoelectric point, and
Modify isoelectric point to (for example):
   reduce the need for enzymes during the cheese production process,
   to allow coagulation of caseins and separation from the Whey proteins during curd formation,
   facilitate production of dairy beverages at lower pH, and
   facilitate coagulation at higher pH to form semi-solid, gel-like dairy alternative products

The modified sequences are shown in Table 3 below, indicating: the modifications made, the predicted isoelectric point, and the rationale behind the design.

Protein isoelectric point was predicted in-silico using IPC2.0 website: http://www.ipc2-isoelectric-point.org/. (Kozlowski, L. P., 2021. IPC 2.0: prediction of isoelectric point and pKa dissociation constants. Nucleic Acids Research).

TABLE 3
Modified dairy protein design

| SEQ | | Amino acid changes | Calculated | |
|---|---|---|---|---|
| ID | | over native beta | isoelectric | |
| NO: | Protein sequence | casein | point | Rationale |
| 1 | RELEELNVPGEIVESLSSSEESITRINKKI | | 5.11 | Native beta-casein |
| | EKFQSEEQQQTEDELQDKIHPFAQTQ | | | |
| | SLVYPFPGPIPNSLPQNIPPLTQTPVVV | | | |
| | PPFLQPEVMGVSKVKEAMAPKHKEM | | | |
| | PFPKYPVEPFTESQSLTLTDVENLHLPL | | | |
| | PLLQSWMHQPHQPLPPTVMFPPQSV | | | |
| | LSLSQSKVLPVPQKAVPYPQRDMPIQ | | | |
| | AFLLYQEPVLGPVRGPFPIIV | | | |
| 2 | RELEELNVPGEIVESLDSDEESITRINKKI | S17D, S19D, S35D | 4.88 | Replacing 3 out of 5 |
| | EKFQDEEQQQTEDELQDKIHPFAQTQ | | | experimentally |
| | SLVYPFPGPIPNSLPQNIPPLTQTPVVV | | | confirmed S |
| | PPFLQPEVMGVSKVKEAMAPKHKEM | | | residues with D |
| | PFPKYPVEPFTESQSLTLTDVENLHLPL | | | residues, to allow |
| | PLLQSWMHQPHQPLPPTVMFPPQSV | | | calcium binding, |
| | LSLSQSKVLPVPQKAVPYPQRDMPIQ | | | and micelle |
| | AFLLYQEPVLGPVRGPFPIIV | | | formation, when |
| | | | | expressed in |
| | | | | heterologous |
| | | | | systems |
| 3 | RELEELNVPGEIVEDLEDEEESITRINKK | S15D, S17E, S18D, | 4.8 | Replacing all 5 |
| | IEKFQDEEQQQTEDELQDKIHPFAQT | S19E, S35D | | experimentally |
| | QSLVYPFPGPIPNSLPQNIPPLTQTPVV | | | confirmed S |
| | VPPFLQPEVMGVSKVKEAMAPKHKE | | | residues with D or E |
| | MPFPKYPVEPFTESQSLTLTDVENLHL | | | residues, to allow |
| | PLPLLQSWMHQPHQPLPPTVMFPPQ | | | calcium binding, |
| | SVLSLSQSKVLPVPQKAVPYPQRDMPI | | | and micelle |
| | QAFLLYQEPVLGPVRGPFPIIV | | | formation, when |
| | | | | expressed in |
| | | | | heterologous |
| | | | | systems |
| 4 | RELEELNVPGEIVEDLDDDEESITRINK | S15D, S17D, S18D, | 4.77 | Replacing all 5 |
| | KIEKFQDEEQQQTEDELQDKIHPFAQT | S19D, S35D | | experimentally |
| | QSLVYPFPGPIPNSLPQNIPPLTQTPVV | | | confirmed S |
| | VPPFLQPEVMGVSKVKEAMAPKHKE | | | residues with D |
| | MPFPKYPVEPFTESQSLTLTDVENLHL | | | residues, to allow |
| | PLPLLQSWMHQPHQPLPPTVMFPPQ | | | calcium binding, |
| | SVLSLSQSKVLPVPQKAVPYPQRDMPI | | | and micelle |
| | QAFLLYQEPVLGPVRGPFPIIV | | | formation, when |
| | | | | expressed in |
| | | | | heterologous |
| | | | | systems, and to |
| | | | | reduce isoelectric |
| | | | | point |
| 38 | RELEELNVPGEIVEDLEDEEESITRINKK | S15D, S17E, S18D, | 4.57 | Replacing all 5 |
| | IEKFQDEEQQQDEDELQDKIHPFAQD | S19E, S35D, T41D, | | experimentally |
| | QSLVYPFPGPIPNSLPQNIPPLTQTPVV | T55D, T154D, | | confirmed S |
| | VPPFLQPEVMGVSKVKEAMAPKHKE | S164E, S166D, | | residues with D/E |
| | MPFPKYPVEPFTESQSLTLTDVENLHL | S168E | | residues and |
| | PLPLLQSWMHQPHQPLPPDVMFPPQ | | | additional predicted |
| | SVLELDQEKVLPVPQKAVPYPQRDMP | | | phosphorylated S/T |
| | IQAFLLYQEPVLGPVRGPFPIIV | | | sites replaced with |
| | | | | D/E, to allow |
| | | | | calcium binding, |
| | | | | when expressed in |
| | | | | heterologous |
| | | | | systems, and to |
| | | | | reduce isoelectric |
| | | | | point |
| 41 | RPKHPIKHQGLPQEVLNENLLRFFVAP | | 4.88 | Bovine (Bos taurus) |
| | FPEVFGKEKVNELSKDIGSESTEDQAM | | | αs1-casein: native |
| | EDIKQM | | | |
| | EAESISSSEEIVPNSVEQKHIQKEDVPS | | | |
| | ERYLGYLEQLLRLKKYKVPQLEIVPNSA | | | |
| | EERL | | | |
| | HSMKEGIHAQQKEPMIGVNQELAYFY | | | |
| | PELFRQFYQLDAYPSGAWYYVPLGTQ | | | |
| | YTDAPSFS | | | |
| | DIPNPIGSENSEKTTMPLW | | | |
| 42 | RPKHPIKHQGLPQEVLNENLLRFFVAP | S46D, S48D, S64D, | 4.70 | Bovine (Bos Taurus) |
| | FPEVFGKEKVNELSKDIGDEDTEDQA | S66D, S67E | | αs1-casein: partial |
| | MEDIKQMEAESIDESEEIVPNSVEQKH | | | replacement of |
| | IQKEDVPSERYLGYLEQLLRLKKYKVPQ | | | phosphorylation |
| | LEIVPNSAEERLHSMKEGIHAQQKEP | | | residues in the two |
| | MIGVNQELAYFYPELFRQFYQLDAYPS | | | main |
| | GAWYYVPLGTQYTDAPSFSDIPNPIGS | | | phosphorylation |
| | ENSEKTTMPLW | | | centers |
| 43 | RPKHPIKHQGLPQEVLNENLLRFFVAP | S41D, S46D, S48D, | 4.58 | Bovine (Bos Taurus) |
| | FPEVFGKEKVNELDKDIGDEDTEDQA | S64D, S66D, S67E, | | αs1-casein: all |
| | MEDIKQMEAEDIDEDEEIVPNSVEQK | S68D | | phosphorylation |
| | HIQKEDVPSERYLGYLEQLLRLKKYKVP | | | residues in the two |
| | QLEIVPNSAEERLHSMKEGIHAQQKEP | | | main |
| | MIGVNQELAYFYPELFRQFYQLDAYPS | | | phosphorylation |
| | GAWYYVPLGTQYTDAPSFSDIPNPIGS | | | centers modified to |
| | ENSEKTTMPLW | | | D/E |
| 44 | RPKHPIKHQGLPQEVLNENLLRFFVAP | S41D, S46D, S48D, | 4.51 | Bovine (Bos Taurus) |
| | FPEVFGKEKVNELDKDIGDEDTEDQA | S64D, S66D, S67E, | | αs1-casein-all |
| | MEDIKQMEAEDIDEDEEIVPNDVEQK | S68D, S75D, S115D | | phosphorylation |
| | HIQKEDVPSERYLGYLEQLLRLKKYKVP | | | residues modified |
| | QLEIVPNDAEERLHSMKEGIHAQQKE | | | to D/E |
| | PMIGVNQELAYFYPELFRQFYQLDAYP | | | |
| | SGAWYYVPLGTQYTDAPSFSDIPNPIG | | | |
| | SENSEKTTMPLW | | | |
| 45 | QEQNQEQPIRCEKDERFFSDKIAKYIPI | | 5.86 | Bovine (Bos Taurus) |
| | QYVLSRYPSYGLNYYQQKPVALINNQF | | | Native kappa-casein |
| | LPYPYYAKPAAVRSPAQILQWQVLSN | | | |
| | TVPAKSCQAQPTTMARHPHPHLSFM | | | |
| | AIPPKKNQDKTEIPTINTIASGEPTSTPT | | | |
| | IEAVESTVATLEASPEVIESPPEINTVQV | | | |
| | TSTAV | | | |
| 46 | QEQNQEQPIRCEKDERFFSDKIAKYIPI | S127D, S149D, | 5.15 | Bovine (Bos Taurus) |
| | QYVLSRYPSYGLNYYQQKPVALINNQF | S166D | | kappa-casein |
| | LPYPYYAKPAAVRSPAQILQWQVLSN | | | Replacing the 3 |
| | TVPAKSCQAQPTTMARHPHPHLSFM | | | phosphorylation |
| | AIPPKKNQDKTEIPTINTIADGEPTSTPT | | | residues with |
| | IEAVESTVATLEADPEVIESPPEINTVQ | | | altered D/E residues |
| | VTDTAV | | | |

### Example 2—Transient Expression of Modified Dairy Proteins in Tobacco Plants

All coding sequences in this work were optimized for expression in the desired target plant (e.g. tobacco, soybean, canola, oat etc.). The optimized gene sequences were chemically synthesized with desired signal peptides, promoters, and terminators to establish the ‘expression cassette’.

The complete expression cassettes (promoter, coding region, and terminator) were cloned in the multiple cloning site (HindIII) of the pCAMBIA0390 plant transformation vectors (Hajdukiewicz, P., Svab, Z. and Maliga, P., 1994. The small, versatile pPZP family of Agrobacterium binary vectors for plant transformation. Plant molecular biology, 25 (6), pp. 989-994).

All protein deigns were fused to transit peptide, HIS tag and Enterokinase site (DDDDK) from the N-terminal end of the protein.

To identify the signal peptide most efficient for beta-casein storage/accumulation, constructs was first transiently expressed in N. tabacum cv. Samsun plants. Two transit peptides were tested in conjugation to the protein designs:
1) Vacuole signal sequence of the barley gene for thiol protease aleurain precursor (Uniprot accession #P05167) MAHARVLLLALAVLATAAVAVASSSSFADSNPIRPVTDRAASTLAQL (SEQ ID NO:39).
2) Apoplast signal sequence of Arabidopsis thaliana endo-1,4-beta-glucanase (Uniprot accession #Q9CAC1) MARKSLIFPVILLAVLLFSPPIYS (SEQ ID NO:40). An ER retention site (KDEL) was fused to the C terminal end of the designed proteins.

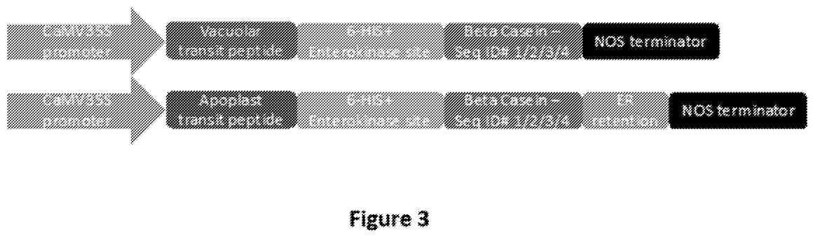

A total of eight binary vectors were synthesized differs in the beta-casein protein design and signal peptide (see FIG. 3 for the schematically represented structure and Table 4 for the construct list).

TABLE 4
List of beta-casein constructs for transient expression

| Construct # | Signal Peptide | Protein design |
|---|---|---|
| 1 | Vacuole | SEQ ID NO: 1 |
| 2 | Apoplast + ER retention site | SEQ ID NO: 1 |
| 3 | Vacuole | SEQ ID NO: 2 |
| 4 | Apoplast + ER retention site | SEQ ID NO: 2 |
| 5 | Vacuole | SEQ ID NO: 3 |
| 6 | Apoplast + ER retention site | SEQ ID NO: 3 |
| 7 | Vacuole | SEQ ID NO: 4 |
| 8 | Apoplast + ER retention site | SEQ ID NO: 4 |

The binary vectors transformed into Agrobacterium tumefaciens GV3101. N. tabacum cv. Samsun plants were grown in the greenhouse for 7 weeks. Consequently, leaves were collected and syringe-infiltrated with different Agrobacterium tumefaciens GV3101 each transformed with one of the eight binary vectors. Different beta-casein protein design expressions were examined 5 days post-inoculation, by Western immunoblotting.

Briefly, fresh leaves were ground with extraction buffer (50 mM Tris HCl PH=7.5, 50 mM NaCl). Following centrifugation, the supernatant was collected, heated to 80° C. and incubated with Ni-NTA beads. Elution from the beads was performed with 250 mM imidazole in extraction buffer. The isolated proteins were precipitated using acetone and uploaded into SDS-PAGE. Beta casein detected using anti beta-casein antibodies (Rabbit polyclonal anti-Casein ab166596 and Goat Anti-Rabbit IgG H&L (HRP) ab205718, Abcam).

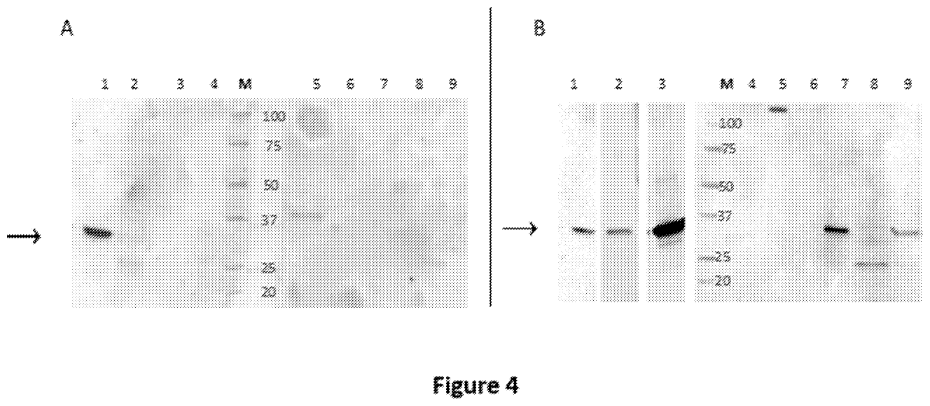

FIG. 4(A) shows representative leaf samples transformed with beta-caseins fused to the vacuolar signal peptide; Lane 1-Positive control (beta-casein 0.5 μg), Lane 2-4: WT; Lane M: protein ladder; Lane 5-9: extract from leaves transformed with constructs 1, 3, 5 and 7 respectively. Bands corresponding to beta caseins are marked with an arrow.

FIG. 4(B) shows representative leaf samples transformed with beta-caseins fused to the apoplast signal peptide with ER retention site at the C-terminal end of the proteins; Lane 1-Positive control (beta-casein 0.5 μg), Lane 2-3: extract from leaves transformed with constructs 2 and 4; Lane M: protein ladder; Lane 4-6: WT; Lane 7: extract from leaves transformed with construct 6; Lane 8-9: extracts from leaves transformed with construct 8. Bands corresponding to beta caseins are marked with an arrow.

Beta-caseins with the vacuolar signal peptide yielded low expression (see FIG. 4A). Significantly higher levels of beta-caseins accumulated when casein was targeted to the ER (see FIG. 4B).

### Example 3—Characterization of Modified Beta-Casein Proteins

Transformation into E. coli

To characterize the different beta casein protein designs we cloned the genes encoding the different beta caseins listed in Table 1 between the Ncol and Xhol sites of pET28a (+) vector, adding an N-terminal hexa-histidine tag. A total of 5 vectors were produced and transformed into E. Coli strain BL21 (DE3). The transformed BL21 (DE3) were incubated at 37° C. to form single colonies. One of the grown single colonies was chosen to grow overnight in 10 mL LB medium containing 50 μg/mL kanamycin at 37° C. 9.5 ml of the starter grown in 0.5 L LB media (in 2 L container) supplemented with 50 μg/mL kanamycin until the OD600 reached 0.5-0.6. Protein synthesis was induced with 0.4 mM β-d-1-thiogalactopyranoside (IPTG) for 2 h at 37° C. The samples were centrifuges at 4° C. in 8,000 rpm for 10 min, pellets kept at −80° C. Bacterial cells were named BC1, BC2, BC3, BC4 and BC5 based on the beta casein sequence (SEQ ID NO: 1-4 and 42 respectively).

### Beta Casein Isolation and Detection

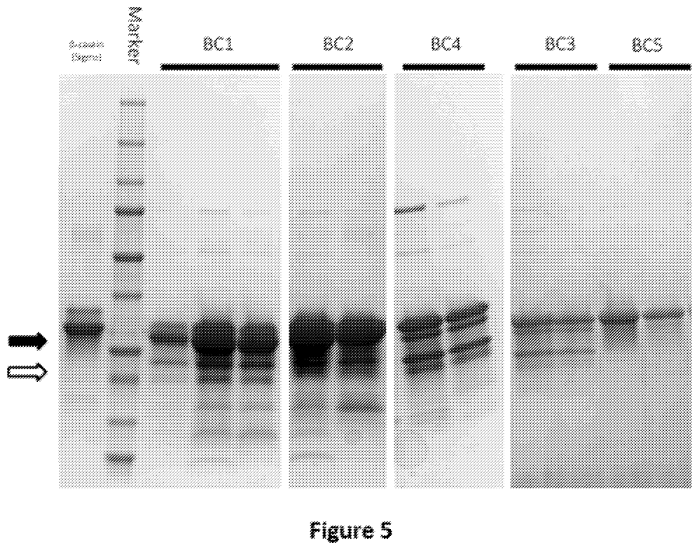

Bacterial cells were resuspended (BC1, BC2, BC3, BC4 and BC5) in 40 mL cooled lysis buffer (50 mM Tris pH-7.5, 0.1% Tween, 2 mM beta-mercaptoethanol, protease inhibitor (AB-ab270055-10, Abcam). Consequently, cells were disrupted using a Sonicator (Sonics vibra cell, Labotal) and centrifuged for 20 minutes at 4° C., 12,000 rpm. The supernatant was heated for 30 min in 80° C. and centrifuged again for 20 min at 12,000 rpm, the consequent supernatant pH was adjusted to pH=5 using HCl. The pellets were resuspended with 10 ml of 50 mM Tris HCl pH 7.5. The resuspended pellets were loaded onto Ni-NTA column (Sepharose Ni-NTA 4 mL) and separated using 250 mM imidazole. Samples from the unbound, wash and different fractions were loaded on SDS-PAGE. The proteins stained using Coomassie blue stain (FIG. 5). The replacement of serines with asparate and glutamate had an impact on protein stability: BC2 which contains three point replacements of serine to aspartate behave as the native casein (BC1), while five replacements of serine amino acids in either aspartate or aspartate and glutamate (BC3 and BC4) had higher proportion of degraded protein than native beta casein. Interestingly BC5 which contained 11 amino acid modifications showed the highest protein stability.

### Thermostability and Secondary Structure of Recombinant Beta-Caseins

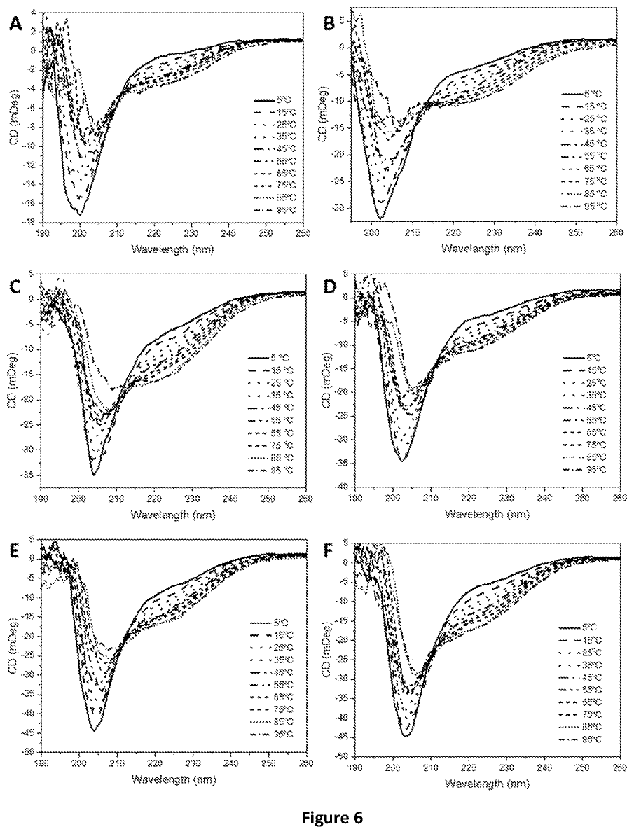

The change in circular dichroism (millidegrees) as a function of wavelength in the far-UV for β-casein at ten different temperatures was examined. Far-UV CD experiments were carried out with 17-20 μM (0.4-0.5 mg/ml) β-casein in a 50 mM Tris-HCl, pH=7.5, 0.1% Tween 20, 2 mM βME. Successive measurements in the far-UV (190-260 nm) were made with overlapping samples at 5-95° C. with 10° C. intervals. For all the beta-casein protein designs tested, we observed an upward shift at 200 nm and a downward shift at 210-230 nm at increased temperatures (FIG. 6). This result suggests increased α-helix structures as the protein was heated. Similar conservation of extended structures and at high temperatures had been previously observed for β-casein by Graham et al. (1984).

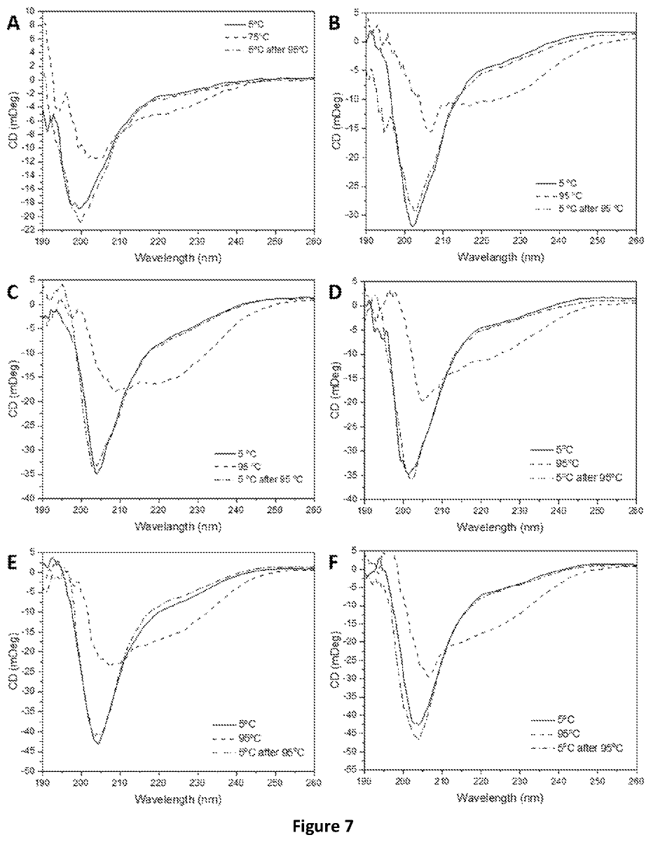

In a different set of experiments, the structural change and recovery following heating to 95° C. and cooling back to 5° C. was examined. All five recombinant β-casein analogues and the bovine beta-casein are stable at 95° C. and maintain their structural arraignment after heating and cooling (FIG. 7). Overall, although the amino acid composition of the casein proteins was changed extensively, the replacements of serine and threonine residues with aspartate and glutamate did not change the thermal behavior of the protein.

### Introducing Negatively Charged Amino Acids Reduce pH Precipitation of Beta-Casein

The precipitation of β-casein at different pH was assessed. The adjustment of the buffers was made using stock solutions of 0.1M Citric acid monohydrate and 0.2M Na₂HPO₄. Samples of 2.5 μg of the six recombinant β-casein analogues and the bovine beta-casein in 100 μL were made by diluting 1 μg/μL stocks in DDW into the different buffers. The solutions were incubated for 30-60 min and centrifuge at 12,000 rpm for 20 min for separation of the soluble and insoluble fractions.

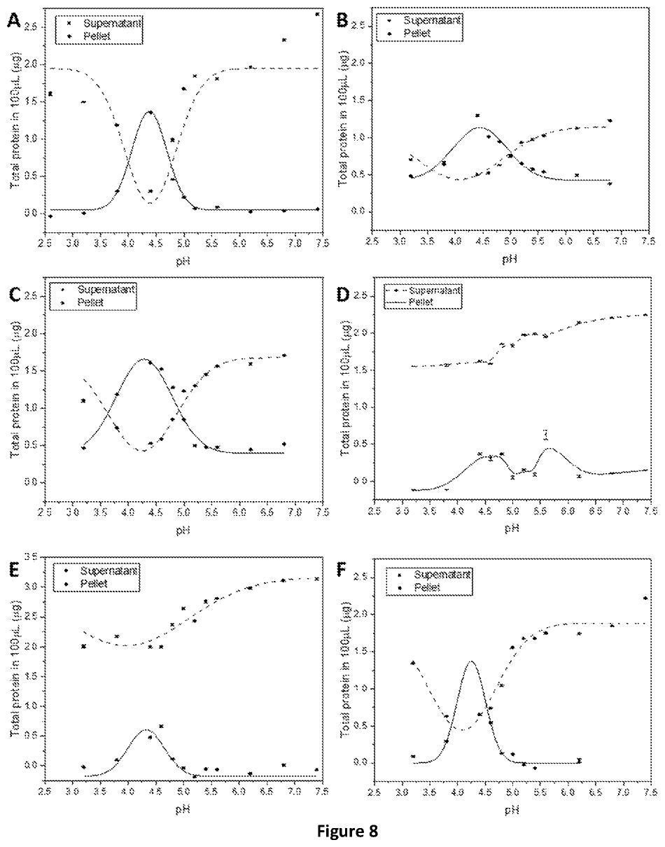

To determine the amount of total protein, Bradford analysis was performed (Bradford absorbance at 595 nm was measured by Synergy H1—Biotek—plate reader). Triplicates of each supernatant and pellet of each protein at the different pH were measured and the amount of protein was calculated using calibration curve of BSA (FIG. 8). The Data was fitted to a normal distribution (eq. 1) using “Origin” software. The fitted results are presented (Table 5).

$$y = {y}_{0} + {Ae}^{- \frac{{( x - {x}_{c} )}^{2}}{2 {w}^{2}}} \qquad Eq . l$$

$${y}_{0} - Offset ; A = Amplitude ; {x}_{c} - center ; w - width$$

BC3 and BC4 did not precipitate at any pH, apparently due to the relatively high proteolytic impurities in the protein samples. The native beta casein (BC1) precipitated at the highest pH (4.44) while all the modified proteins (BC2 and 5) precipitated at lower pH (4.25-4.28).

TABLE 5
pH precipitation points of Beta casein analogues

| Protein | pH Pellet |
|---|---|
| BC1 - control | 4.44 ± 0.06 |
| BC2 | 4.28 ± 0.01 |
| BC5 | 4.25 ± 0.02 |

### Enhanced Calcium Binding of the Modified Dairy Proteins

To measure the calcium binding to beta-caseins, the Isothermal titration calorimetry (ITC) method was used. The different casein proteins were Incubated with 10 mM of CaCl₂) or β-Tricalcium Phosphate for 1 hour at room temperature. Following incubation the proteins passed through dialysis overnight (dialysis buffer: 50 mM Tris HCl pH=7.5, 0.1% Tween-20, 2 mM beta-mercaptoethanol, protease inhibitor cocktail; sample: dialysis buffer volume ratio of at least 1:500). The beta-caseins were titrated using the dialysis buffer+EGTA (0.5 mM EGTA titration into 25 μM β-casein of various sources). The measurements were performed by MicroCal PEAQ-ITC.

The calorimetric titration curves showed little if any heat evolution or heat consumption for the reaction between BC1 (native beta-casein) and CaCl₂). BC3 and BC5 consumed heat upon reaction with CaCl₂) indicating endothermic binding of Ca2+ to the modified beta-caseins.

### Example 4—Self-Association and Micelle Formation of the Modified Dairy Proteins

The self-association properties of recombinant β-casein analogues, bovine beta-casein, and dephosphorylated beta-casein can be determined. Generally, β-casein self-association refers to the process in which β-casein monomers spontaneously form micelles through a balance of hydrophobic and electrostatic interactions. Dynamic light-scattering (Zetasizer Nano ZS, Malvern Instruments Ltd, equipped with a temperature controlled small-volume cell holder) can be used to the monitor changes in particle size, which is as an important indicator of protein aggregation (Crowley et al., 2019).

The self-association of β-casein is known to be influenced by protein concentration, pH and temperature, or the presence of calcium (Li et al., 2019). How the recombinant modified β-caseins undergo self-association at a range of protein concentrations, temperatures, and calcium ion concentrations can be determined. It would be expected that introduction of negative charges by aspartate and glutamate residues will enhance calcium binding, as indicated in the previous example, and thus will promote beta-casein aggregation in the presence of calcium. The critical micelle concentration (CMC) of the modified β-casein proteins, bovine beta-casein, and dephosphorylated beta-casein can be determined by fluorescence spectrophotometry using pyrene as fluorescent probe, as described recently (Song et al, 2023). CMC is defined as the concentration of protein above which small aggregates are formed. The intensity ratio (I1/I3) of the first and third vibronic peak in the emission spectrum of pyrene varies with environmental hydrophobicity, which is affected by the aggregation of β-casein. When β-casein is monomer, pyrene is exposed to the aqueous environment of β-CN solution and the I1/I3 is high. However, pyrene enters the hydrophobic microregion of aggregates from the aqueous phase when aggregates are formed and thereby the I1/I3 decreases significantly. The β-CN concentration corresponding to this change is the CMC value. Generally, the lower the CMC value of a protein, the more easily the protein will aggregate (Li et al., 2019). The effect of temperature and calcium addition on CMC will also be determined. The knowledge gained will allow development of protein aggregates/micelles from recombinant β-casein analogues, with different particle sizes and stability.

### Example 5—Production of Animal-Free Milk Using the Modified Dairy Proteins

The self-association and emulsification properties of modified beta-caseins can be leveraged to produce an animal-free milk and mimic flavor and mouthfeel of bovine milk. In principle, it is expected that modified recombinant beta-caseins with similar calcium-binding ability to native beta-casein will self-associate into aggregates at the critical micelle concentration (CMC), calcium concentration, pH and temperature, determined in Example 4. Selecting the self-association conditions of modified beta-caseins at pH ˜6.7 (pH of milk), can enable formation of micellar/aggregate suspensions to which other ingredients, such as sugars, minerals, and fat droplets can be added.

The excellent emulsifying properties of bovine beta-casein are well-recognized and they refer to the amount of oil that can be emulsified by a unit weight of the protein (Atamer et al., 2017). Monomeric modified beta-caseins can be used as emulsifiers to prepare an oil-in-water emulsion using homogenization (˜200 bar). Subsequently, micellar suspensions can be mixed with oil-in-water emulsions and other water-soluble ingredients by high-shear mixing to produce a milk-like product. To compare the performance of modified beta caseins, animal-free milk using bovine beta-casein (from Sigma Aldrich) can be prepared, followed by analysis of the physicochemical properties of the final product, such as pH, viscosity, color and physical stability (particle size and zeta potential).

The typical composition of bovine milk is 3.2. % protein, 3.3% fat, 5.3% lactose, and 0.7% minerals (Haug et al., 2007). Thus, the composition of the animal-free milk can be produced with a similar composition to bovine milk as outline in Table 6 below:

TABLE 6
Theorical nutrient composition of animal-free milk.

| Components | Percentage (%) |
|---|---|
| Monomeric beta casein | 0.1 |
| Micellar beta casein | 3.1 |
| Coconut oil | 3.3 |
| Mineral salts (including | 0.7 |
| Calcium) | |
| Cane sugar | 5.3 |
| Dipotassium phosphate | 0.05 |
| Sodium phosphate | 0.05 |
| Water | 87.4 |

Other modified dairy proteins of the invention, including but not limited to those in Table 3 and others described herein, can of course be expressed, tested and processed, as described herein including as described in Examples 2 to 5.

### REFERENCES

Blom et al., 1999; Blom, N., Gammeltoft, S. and Brunak, S., 1999. Sequence and structure-based prediction of eukaryotic protein phosphorylation sites. Journal of molecular biology, 294 (5), pp. 1351-1362
Graham et al., 1984; Graham, E. R. B., Malcolm, G. N. and Mckenzie, H. A., 1984. On the isolation and conformation of bovine β-casein A1. International Journal of Biological Macromolecules 6.3, pp. 155-161.
Huppertz et al., 2018; Huppertz, T., Fox, P. F. and Kelly, A. L., 2018. The caseins: Structure, stability, and functionality. In Proteins in food processing pp. 49-92
Kozlowski L P (2021); Kozlowski, L. P., 2021. IPC 2.0: prediction of isoelectric point and pKa dissociation constants. Nucleic Acids Research.
Philip et al., 2001; Philip, R., Darnowski, D. W., Maughan, P. J. and Vodkin, L. O., 2001. Processing and localization of bovine β-casein expressed in transgenic soybean seeds under control of a soybean lectin expression cassette. Plant Science, 161 (2), pp. 323-335
Li, M., Auty, M. A. E., Crowley, S. V., Kelly, A. L., O'Mahony, J. A., & Brodkorb, A. (2019). Self-association of bovine beta-casein as influenced by calcium chloride, buffer type and temperature. Food Hydrocolloids, 88, 190-198. https://doi.org/10.1016/j.foodhyd.2018.09.035
Song, S., Lin, Y, Zhang, Y, Luo, Y and Guo, H. (2023. The self-association properties of partially dephosphorylated bovine beta-casein, Food Hydrocolloids, Volume 134, 1-11.
Haug, A., Høstmark, A. T. & Harstad, O. M. Bovine milk in human nutrition—a review. Lipids Health Dis 6, 25 (2007). https://doi.org/10.1186/1476-511X-6-25
Atamer, Z., Post, A. E., Schubert, T., Holder, A., Boom, R. M., & Hinrichs, J. (2017). Bovine β-casein: Isolation, properties and functionality. A review. International Dairy Journal, 66, 115-125. https://doi.org/10.1016/j.idairyj.2016.11.010

1. A modified dairy protein in which at least one potentially phosphorylated amino acid has been replaced with a negatively charged amino acid.

2. The modified dairy protein of claim 1 in which the potentially phosphorylated amino acid is selected from serine(S) and threonine (T).

3. The modified dairy protein of any preceding claim in which the negatively charged amino acid is selected from aspartate (D) and glutamate (E).

4. The modified dairy protein of any preceding claim which when recombinantly expressed in a heterologous expression system, exhibits at least one of the following:
a) capacity to bind calcium, and
b) capacity to form micelles.

5. The modified dairy protein of any preceding claim which when recombinantly expressed in a heterologous expression system, exhibits at least one of the following:
a) an improved capacity to binding calcium relative to the unmodified dairy protein when it is recombinantly expressed in the same heterologous expression system,
b) an improved capacity to form a micelle, relative to the unmodified dairy protein when it is recombinantly expressed in the same heterologous expression system,
c) an altered pl relative to that of the unmodified dairy protein when it is recombinantly expressed in the same heterologous expression system
d) altered thermal stability relative to that of the unmodified dairy protein when it is recombinantly expressed in the same heterologous expression system
e) an altered zeta potential relative to that of the unmodified dairy protein when it is recombinantly expressed in the same heterologous expression system.

6. The modified dairy protein of any preceding claim which apart from the modifications described is otherwise the same in sequence as the unmodified dairy protein.

7. A polynucleotide encoding the modified dairy protein of any preceding claim

8. An expression construct comprising the polynucleotide of claim 7

9. A host cell, plant cell, organism, plant, plant part, plant tissue, propagule, progeny, or seed, comprising at least one of:
a) a dairy protein of claim 1,
b) polynucleotide of claim 7 and
c) construct of claim 8.

10. The host cell, plant cell, organism, or plant of claim 9 that is heterologous to the species from which the unmodified dairy protein is derived.

11. A method for producing a modified dairy protein of any preceding claim, the method comprising expressing a polynucleotide encoding the modified dairy protein in a host cell, plant cell, organism, plant, plant part, plant tissue, propagule, progeny, or seed.

12. The method of claim 11 comprising the step of testing the expressed modified dairy protein with respect to at least one of its:
a) capacity to bind calcium,
b) capacity to form a micelle,
c) pl,
d) thermal stability, and
e) zeta potential

13. The method of claim 12 in which the modified dairy protein is selected based on at least one of a)-e).

14. The method of any preceding claims comprising the step of purifying the modified dairy protein from the host cell, plant cell, organism, plant, plant part, plant tissue, propagule, progeny, or seed.

15. A modified dairy protein produced by the method of any preceding claim.

16. A food or beverage product or ingredient comprising one or more modified dairy protein of any preceding claim or produced by the method of any preceding claim.

17. A method for producing a food or beverage product or ingredient, the method including the step of processing or incorporating the modified dairy protein of any preceding claim, or produced by the method of any preceding claim, into food or beverage product or ingredient.

18. Use of the modified dairy protein of any preceding claim, or produced by the method of any preceding claim, in preparation of a food or beverage product or ingredient.

19. A food or beverage product or ingredient produced by the method of claim 17.

20. The food or beverage product or ingredient of claim 16 or 19 that is selected from milk, cream, chocolate, butter, cheese, fermented yoghurt, ice-cream, infant formula, protein beverages, custards, buttermilk, milk powders, margarine, whey-protein concentrates and isolates, milk protein concentrates or isolates and hydrolysed milk proteins.

21. The food or beverage product or ingredient of claim 16 or 19 that is a dairy product substitute.

22. The food or beverage product or ingredient of claim 16 or 19 that is a dairy product substitute selected from a milk substitute, a cream substitute, a whipping cream substitute, a mayonnaise substitute, an ice cream substitute, a cheese substitute, and a yoghurt substitute.


SEQUENCE LISTING (52 sequences)

SEQ ID NO: 1 - protein, 209 aa, Bos taurus
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 2 - protein, 209 aa, synthetic construct
RELEELNVPGEIVESLDSDEESITRINKKIEKFQDEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 3 - protein, 209 aa, synthetic construct
RELEELNVPGEIVEDLEDEEESITRINKKIEKFQDEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 4 - protein, 209 aa, synthetic construct
RELEELNVPGEIVEDLDDDEESITRINKKIEKFQDEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 5 - DNA, 627 bp, Bos taurus
  source 1..627: mol_type = other DNA
agagagctggaagaactcaatgtaccgggtgagattgtggaaagcctttcaagcagtgaggaatctattacacgcatcaataagaaaattgagaagtttcagagtgaggaacagcagcaaacagaggatgaactccaggataaaatccacccctttgcccagacacagtctctagtctatcccttccctgggcccatccctaacagcctcccacaaaacatccctcctcttactcaaacccctgtggtggtgccgcctttccttcagcctgaagtactgggagtctccaaagtgaaggaggctatggctcctaagcacaaagaaatgccctttcctaaatatccagttgagccctttactgaaagccagagcttgactttgactgatgttgaaaatcttcatttgcctcctctcttgctccagtcttggatgcatcagcctcaccagcctcttcctccaactgtcatgtttcctcctcagtccgtgctgtccctttctcagtccaaagtcctgcctgttccccagaaagcagtgccctatccccagagagatatgcccattcaggcctttctgctgtaccagcagcctgtactcggtcctgtccggggacctttccctattattgtc

SEQ ID NO: 6 - protein, 199 aa, Bos taurus
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 7 - DNA, 600 bp, Bos taurus
  source 1..600: mol_type = other DNA
aggcctaaacatcctatcaagcaccaaggactccctcaagaagtcctcaatgaaaatttactcaggttttttgtggcaccttttccagaagtgtttggaaaggagaaggtcaatgaactgagcaaggatattgggagtgaatcaactgaggatcaagccatggaagatattaagcaaatggaagctgaaagcatttcgtcaagtgaggaaattgttcccaatagtgttgagcagaagcacattcaaaaggaagatgtgccctctgagcgttacctgggttatctggaacagcttctcagactgaaaaaatacaaagtaccccagctggaaattgttcccaatagtgctgaggaacgacttcacagtatgaaagagggaatccatgcccaacagaaagaacctatgataggagtgaatcaggaactggcctacttctaccctgagcttttcagacaattctaccagctggatgcctatccatctggtgcctggtattacgttccactaggcacacaatacactgatgccccatcattctctgacatccctaatcctattggctctgagaacagtgaaaagactactatgccactgtggtga

SEQ ID NO: 8 - protein, 207 aa, Bos taurus
KNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 9 - protein, 621 aa, Bos taurus
AAGAATACGATGGAACATGTCTCCTCCAGTGAGGAATCTATCATCTCCCAGGAAACATATAAGCAGGAAAAGAATATGGCCATTAATCCCAGCAAGGAGAACCTTTGCTCCACATTCTGCAAGGAAGTTGTAAGGAACGCAAATGAAGAGGAATATTCTATCGGCTCATCTAGTGAGGAATCTGCTGAAGTTGCCACTGAGGAAGTTAAGATTACTGTGGACGATAAGCACTACCAGAAAGCACTGAATGAAATCAATCAGTTTTATCAGAAGTTCCCCCAGTATCTCCAGTATCTGTATCAAGGTCCAATTGTTTTGAACCCATGGGATCAGGTTAAGAGGAATGCTGTTCCCATTACTCCCACTCTGAACAGAGAGCAGCTCTCCACCAGTGAGGAAAATTCAAAGAAGACCGTTGACATGGAATCAACAGAAGTATTCACTAAGAAAACTAAACTGACTGAAGAAGAAAAGAATCGCCTAAATTTTCTGAAAAAAATCAGCCAGCGTTACCAGAAATTCGCCTTGCCCCAGTATCTCAAGACTGTTTATCAGCATCAGAAAGCTATGAAGCCATGGATTCAACCTAAGACAAAGGTTATTCCCTATGTGAGGTACCTT

SEQ ID NO: 10 - protein, 209 aa, Bos taurus
RELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 11 - DNA, 627 bp, Bos taurus
  source 1..627: mol_type = other DNA
agagagctggaagaactcaatgtacctggtgagattgtggaaagcctttcaagcagtgaggaatctattacacgcatcaataagaaaattgagaagtttcagagtgaggaacagcagcaaacagaggatgaactccaggataaaatccacccctttgcccagacacagtctctagtctatcccttccctgggcccatccataacagcctcccacaaaacatccctcctcttactcaaacccctgtggtggtgccgcctttccttcagcctgaagtaatgggagtctccaaagtgaaggaggctatggctcctaagcacaaagaaatgcccttccctaaatatccagttgagccctttactgaaagccagagcctgactctcactgatgttgaaaatctgcaccttcctctgcctctgctccagtcttggatgcaccagcctcaccagcctcttcctccaactgtcatgtttcctcctcagtccgtgctgtccctttctcagtccaaagtcctgcctgttccccagaaagcagtgccctatccccagagagatatgcccattcaggcctttctgctgtaccaggagcctgtactcggtcctgtccggggacccttccctattattgtc

SEQ ID NO: 12 - protein, 169 aa, Bos taurus
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 13 - DNA, 510 bp, Bos taurus
  source 1..510: mol_type = other DNA
caggagcaaaaccaagaacaaccaatacgctgtgagaaagatgaaagattcttcagtgacaaaatagccaaatatatcccaattcagtatgtgctgagcaggtatcctagttatggactcaattactaccaacagaaaccagttgcactaattaataatcaatttctgccatacccatattatgcaaagccagctgcagttaggtcacctgcccaaattcttcaatggcaagttttgtcaaatactgtgcctgccaagtcctgccaagcccagccaactaccatggcacgtcacccacacccacatttatcatttatggccattccaccaaagaaaaatcaggataaaacagaaatccctaccatcaataccattgctagtggcgagcctacaagtacacctaccaccgaagcagtagagagcactgtagctactctagaagattctccagaagttattgagagcccacctgagatcaacacagtccaagttacttcaactgcagtctaa

SEQ ID NO: 14 - protein, 199 aa, Capra hircus
RPKHPINHRGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 15 - DNA, 600 bp, Capra hircus
  source 1..600: mol_type = other DNA
aggcctaaacatcctatcaatcaccaaggactctctccagaagtcctcaatgaaaatttactcaggtttgttgtggcgccttttccagaagtgtttagaaaggagaacatcaatgaactgagtaaggatattgggagtgaatcaactgaggatcaagccatggaagatgctaagcaaatgaaagctggaagcagttcgtcaagtgaggaaattgttcccaatagtgctcagcagaagtacattcaaaaggaagatgtgccctctgagcgttacctgggttatctggaacagcttctcagactgaaaaaatacaacgtgccccagctggaaattgttcccaaaagtgctgaggaacaacttcacagtatgaaagagggaaaccctgcccaccagaaacagcctatgatagcagtgaaccaggaactggcctacttctaccctcagcttttcagacaattctaccagctggatgcctatccatctggtgcctggtactaccttccactaggcacacaatacactgatgccccctcattctctgacatccctaatcccattggctctgagaacagtggaaagactactatgccactgtggtga

SEQ ID NO: 16 - protein, 208 aa, Capra hircus
KHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 17 - DNA, 627 bp, Capra hircus
  source 1..627: mol_type = other DNA
aagcataagatggaacatgtctcctccagtgaggaacctatcaatatcttccaggaaatatataagcaggaaaagaatatggccattcatcccagaaaggagaaactttgcaccacatcctgtgaggaagttgtaaggaacgcaaatgaagaggaatattctatcagatcatctagtgaggaatctgctgaagttgccccagaggaaattaagattactgtggacgataagcactaccagaaagccctgaatgaaatcaatcagttttatcagaagttcccccagtatctccagtatccgtatcaaggtccaattgttttgaacccatgggatcaggttaagagaaatgctggcccctttactcccaccgtgaacagagagcagctctccaccagtgaggaaaattcaaagaagaccattgatatggaatcaacagaagtattcactaagaaaactaaactgactgaagaagaaaagaatcgcctaaattttctgaaaaaaatcagccagtattaccagaaatttgcctggccccagtatctcaagactgttgatcagcatcagaaagctatgaagccatggactcaacctaagacaaatgctattccctatgtgaggtacctttaa

SEQ ID NO: 18 - protein, 207 aa, Capra hircus
REQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGPFPILV

SEQ ID NO: 19 - DNA, 624 bp, Capra hircus
  source 1..624: mol_type = other DNA
agagagcaggaagaactcaatgtagtcggtgagactgtggaaagcctttcaagcagtgaggaatctattacacacatcaataagaaaattgagaagtttcaaagtgaggaacaacagcaaacagaggatgaactccaggataaaatccacccctttgcccaggcacagtctctagtctatcccttcactgggcccatccctaacagcctcccacaaaacatcctgcctcttactcaaacccctgtggtggtgccgcctttccttcagcctgaaataatgggagtccccaaagtgaaggagactatggttcctaagcacaaagaaatgcccttccctaaatatccagttgagccctttactgaaagccagagcctgactctcactgatgttgaaaagctgcaccttcctctgcctctggtccagtcttggatgcaccagcctccccagcctctttctccaaccgtcatgtttcctcctcagtccgtgctgtccctttctcagcccaaagttctgcctgttccccagaaagcagtgccccagagagatatgcccatccaggcctttctgctgtaccaggagcctgtacttggtcctgtccggggacccttccctattcttgtctaa

SEQ ID NO: 20 - protein, 171 aa, Capra hircus
QEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 21 - DNA, 516 bp, Capra hircus
  source 1..516: mol_type = other DNA
caggagcaaaaccaggaacaaccgatatgctgtgagaaagatgaaagattcttcgatgacaaaatagccaaatatatcccaattcagtatgtgctgagtaggtatcctagttatggactcaattactatcaacagagaccagttgcactaattaataatcaatttctgccatacccatattatgcaaagccagttgcagttaggtcacctgcccaaactcttcaatggcaagttttgccaaatactgtgcctgccaagtcctgccaagaccagccaactaccctggcacgtcacccacacccacatttatcatttatggccattccaccaaagaaagatcaggataaaacagaagtccctgccatcaataccattgctagtgctgagcctacagtacacagtacacctaccaccgaagcaatagtgaacactgtagataatccagaagcttcctcagaatcgattgcgagtgcatctgagaccaacacagcccaagttacttcaaccgaggtctaa

SEQ ID NO: 22 - protein, 199 aa, Ovis aries
RPKHPIKHQGLSSEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 23 - DNA, 573 bp, Ovis aries
  source 1..573: mol_type = other DNA
aggcctaaacatcctatcaagcaccaaggactctctccagaagtcctcaatgaaaatttactcaggtttgttgtggcgccttttccagaagtgtttagaaaggagaacatcaatgaactgagtaaggatattgggagtgaatcaattgaggatcaagccatggaagatgctaagcaaatgaaagctggaagcagttcgtcaagtgaggaaattgttcccaatagtgctgagcagaagtacattcaaaaggaagatgtgccctctgagcgttacttgggttatctggaacagcttctcagactgaaaaaatacaacgtgccccagctggaaattgttcccaaaagtgctgaggaacaacttcatagtatgaaagagggaaaccctgcccaccagaaacagcctatgatagcagtgaatcagcttttcagacaattctaccagctggacgcctatccatctggtgcctggtattaccttccactaggcacacaatacactgatgccccctcattctctgacatccctaatcccattggctctgagaacagtggaaagattactatgccactgtgg

SEQ ID NO: 24 - protein, 208 aa, Ovis aries
KHKMEHVSSSEEPINISQEIYKQEKNMAIHPRKEKLCTTSCEEVVRNADEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 25 - DNA, 624 bp, Ovis aries
  source 1..624: mol_type = other DNA
aagcataagatggaacatgtctcctccagtgaggaacctatcaatatctcccaggaaatatataagcaggaaaagaatatggccattcatcccagaaaggagaaactttgcaccacatcctgtgaggaagttgtaaggaacgcagatgaagaggaatattctatcagatcatctagtgaggaatctgctgaagttgccccagaggaagttaagattactgtggacgataagcactaccagaaagcactgaatgaaatcaatcagttttatcagaagttcccccagtacctccagtatctgtatcaaggcccaattgttttgaacccatgggatcaggttaagagaaatgctggcccctttactcccaccgtgaacagagagcagctctccaccagtgaggaaaattcaaagaagaccattgatatggaatcaacagaagtattcactaagaaaactaaactgactgaagaagaaaagaatcgcctaaattttctgaaaaaaatcagccagtattaccagaaatttgcctggccccagtatctcaagactgttgatcagcatcagaaagctatgaagccatggactcaacctaagacaaacgctattccctatgtgaggtacctt

SEQ ID NO: 26 - protein, 207 aa, Ovis aries
REQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLPPTVMFPPQSVLSLSQPKVLPVPQKAVPQRDMPIQAFLLYQEPVLGPVRGPFPILV

SEQ ID NO: 27 - DNA, 624 bp, Ovis aries
  source 1..624: mol_type = other DNA
agagagcaggaagaactcaatgtagtcggtgagactgtggaaagcctttcaagcagtgaggaatctattacacacatcaataagaaaattgagaagtttcaaagtgaggaacaacagcaaacagaggatgaactccaggataaaatccacccctttgcccaggcacagtctctagtctatcccttcactgggcccatccctaacagcctcccacaaaacatcctgcctcttactcaaacccctgtggtggtgccgcctttccttcagcctgaaataatgggagtccccaaagtgaaggagactatggttcctaagcacaaggaaatgcccttccctaaatatccagttgagccctttactgaaagccagagcctgactctcactgatgttgaaaagctgcaccttcctctgcctctggtccagtcttggatgcaccagcctccccagcctcttcctccaaccgtcatgtttcctcctcagtccgtgctgtccctttctcagcccaaagttctgcctgttccccagaaagcagtgccccagagagatatgcccatccaggcctttctgctgtaccaggagcctgtacttggtcctgtccggggacccttccctattcttgtctaa

SEQ ID NO: 28 - protein, 171 aa, Ovis aries
QEQNQEQRICCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 29 - DNA, 516 bp, Ovis aries
  source 1..516: mol_type = other DNA
caggagcaaaaccaagaacaacgaatatgctgtgagaaagatgaaagattcttcgatgacaaaatagccaaatatatcccaattcagtatgtgctgagtaggtatcctagttatggactcaattactaccaacagagaccagttgcactaattaataatcaatttctgccatacccatattatgcgaagccagttgcagttaggtcacctgcccaaactcttcaatggcaagttttgccaaatgctgtgcctgccaagtcctgccaagaccagccaactgccatggcacgtcacccacacccacatttatcatttatggccattccaccaaagaaagatcaggataaaacagaaatccctgccatcaataccattgctagtgctgagcctacagtacacagtacacctaccaccgaagcagtagtgaacgctgtagataatccagaagcttcctcagaatcgattgcgagtgcacctgagaccaacacagcccaagttacttcaaccgaggtctaa

SEQ ID NO: 30 - protein, 215 aa, Camelus dromedarius
RPKYPLRYPEVFQNEPDSIEEVLNKRKILELAVVSPIQFRQENIDELKDTRNEPTEDHIMEDTERKESGSSSSEEVVSSTTEQKDILKEDMPSQRYLEELHRLNKYKLLQLEAIRDQKLIPRVKLSSHPYLEQLYRINEDNHPQLGEPVKVVTQEQAYFHLEPFPQFFQLGASPYVAWYYPPQVMQYIAHPSSYDTPEGIASEDGGKTDVMPQWW

SEQ ID NO: 31 - DNA, 624 bp, Camelus dromedarius
  source 1..624: mol_type = other DNA
aggcctaaatatcctctcaggtacccagaagtctttcaaaatgaaccagacagcatagaggaagtcctcaacaaaagaaagattcttgagttagcagtggtttcacccattcagtttagacaggagaacatcgatgaactgaaggatactaggaacgaaccaaccgaagatcacatcatggaagacactgagcgaaaggaatctggaagcagttcaagtgaggaagttgtttccagtaccactgagcagaaggacattctcaaggaagatatgccctcccaacgctatctggaagagcttcacagactgaacaaatacaaactactccagctggaagctatccgtgaccagaaacttattccaagagtgaagctgtcctcccacccatatctggaacaactttacagaataaatgaggacaaccacccccaactgggggagcctgtgaaagtagtgactcagcctttcccacaattcttccagcttggtgcctctccctatgttgcttggtattatcctccacaagtcatgcaatatattgctcacccctcatcctacgacacccctgaaggcattgcctctgaggacggtggaaaaaccgacgttatgccacagtggtggtaa

SEQ ID NO: 32 - protein, 178 aa, Camelus dromedarius
KHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFTKKTELTEEEKDHQKFLNKIYQYYQTFLWPEYLKTVYQYQKTMTPWNHIKRYF

SEQ ID NO: 33 - DNA, 537 bp, Camelus dromedarius
  source 1..537: mol_type = other DNA
aagcatgagatggatcagggctcctccagtgaggaatctatcaacgtctctcagcaaaaatttaagcaggtaaagaaggtggccattcatcccagcaaggaggacatctgctccacattttgcgaggaagctgtaaggaacataaaggaagtggaatcagctgaagtccccacagagaacaaaatcagtcaattttatcagaagtggaaattcctccagtatctccaggctcttcatcaaggtcagattgtgatgaacccatgggatcagggcaagacaagggcctacccctttattcccactgtgaacacagagcagctctccatcagtgaggaatcaactgaagttcccacagaggaatcaacagaagtattcactaagaaaactgaattgactgaagaagaaaaggatcaccaaaaatttctgaacaaaatctaccaatattatcagacattcctctggccagagtatctcaagactgtttatcaatatcagaaaactatgactccatggaatcacatcaagagatacttttaa

SEQ ID NO: 34 - protein, 217 aa, Camelus dromedarius
REKEEFKTAGEALESISSSEESITHINKQKIEKFKIEEQQQTEDEQQDKIYTFPQPQSLVYSHTEPIPYPILPQNFLPPLQPAVMVPFLQPKVMDVPKTKETIIPKRKEMPLLQSPVVPFTESQSLTLTDLENLHLPLPLLQSLMYQIPQPVPQTPMIPPQSLLSLSQFKVLPVPQQMVPYPQRAMPVQAVLPFQEPVPDPVRGLHPVPQPLVPVIA

SEQ ID NO: 35 - DNA, 654 bp, Camelus dromedarius
  source 1..654: mol_type = other DNA
agagagaaggaagaattcaagacagccggtgaggctttggaaagcatttcaagcagtgaggaatctattacacacatcaacaagcagaaaattgagaagtttaaaattgaggaacagcagcaaacagaggatgaacagcaggataaaatctacacctttccccagccccagtctctagtctattctcacactgagcccatcccttaccctatccttccacaaaactttctgccgcctcttcagcctgctgtgatggtaccttttcttcagcctaaagtaatggatgtccccaaaactaaggagaccatcattcctaagcgcaaagaaatgcccttgcttcagtctccagtagtgccctttactgaaagccagagcctgactctcactgatctcgaaaatctgcaccttcctctgcctctgctccagtctttgatgtaccagattccccagcctgttcctcagacccccatgattcctcctcagtccctgctgtccctttctcagttcaaagtcctgcctgttccccagcaaatggtgccctacccccagagagccatgcctgtgcaagccgttctgcccttccaggagcctgtacctgaccccgtccgggggctccaccctgtgcctcaaccacttgtccctgtgattgcctaa

SEQ ID NO: 36 - protein, 162 aa, Camelus dromedarius
EVQNQEQPTCFEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 37 - DNA, 489 bp, Camelus dromedarius
  source 1..489: mol_type = other DNA
gaggtgcaaaaccaggaacaaccaacatgttttgagaaggtggaaagattgctcaatgaaaaaacagtcaaatatttcccaattcagttcgtgcagagtagatatcctagttatggaatcaattactaccaacacagactagctgtaccaattaataaccaatttattccttacccaaattatgccaagccagttgcaattagactacatgctcaaattcctcagtgccaagccctgccaaatatcgacccacctactgtggaacgtcggccacgcccacgtccatcatttattgccattcccccaaagaaaacccaggataaaacagtcaaccctgccatcaatactgttgctactgttgagcctccagttatacctaccgccgaaccagcagtgaacactgtagttattgcagaagcttcctcagaattcatcaccacaagcacacccgagaccaccacagtccaaattacttcgactgagatctaa

SEQ ID NO: 38 - protein, 209 aa, synthetic construct
RELEELNVPGEIVEDLEDEEESITRINKKIEKFQDEEQQQDEDELQDKIHPFAQDQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPDVMFPPQSVLELDQEKVLPVPQKAVPYPQRDMPIQAFLLYQEPVLGPVRGPFPIIV

SEQ ID NO: 39 - protein, 47 aa, Hordeum vulgare
MAHARVLLLALAVLATAAVAVASSSSFADSNPIRPVTDRAASTLAQL

SEQ ID NO: 40 - protein, 24 aa, Arabidopsis thaliana
MARKSLIFPVILLAVLLFSPPIYS

SEQ ID NO: 41 - protein, 199 aa, Bos taurus; identical to SEQ ID NO: 14

SEQ ID NO: 42 - protein, 199 aa, synthetic construct
RPKHPIKHQGLPQEVLNENLLRFFVAPFPEVFGKEKVNELSKDIGDEDTEDQAMEDIKQMEAESIDESEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 43 - protein, 199 aa, synthetic construct
RPKHPIKHQGLPQEVLNENLLRFFVAPFPEVFGKEKVNELDKDIGDEDTEDQAMEDIKQMEAEDIDEDEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 44 - protein, 199 aa, synthetic construct
RPKHPIKHQGLPQEVLNENLLRFFVAPFPEVFGKEKVNELDKDIGDEDTEDQAMEDIKQMEAEDIDEDEEIVPNDVEQKHIQKEDVPSERYLGYLEQLLRLKKYKVPQLEIVPNDAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 45 - protein, 169 aa, Bos taurus
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 46 - protein, 169 aa, synthetic construct
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIADGEPTSTPTIEAVESTVATLEADPEVIESPPEINTVQVTDTAV

SEQ ID NO: 47 - DNA, 1500 bp, Brassica napus
  source 1..1500: mol_type = genomic DNA
tgctgacttctccagatactcaaatcgaaaatataaagtttggataatgatatttgtcatagatttatgtaaaagtttcaaaaagtccataaaattttaaataactaaaaatcatgttagataacttacaggttaatatatttgataattatcaataagttcatataaattcgtacatatgtaagaccatctccaatagtgctccgtatcttctctataattcccactaaaatagagtaattctattatagagttgattttgctccaatggttaactctataatagagtgaaatatagagtaatgttgttttttttcattctatatttggagtagaaaaacaacattcatctatatttcactctattatagagttgctctatttagtggaaattatagagcaccatgaagatgtcataaatgtacgtggtgtttcctttttccgatcaatttattgttagaccaaaagattaaatataatgtaattgaagtgttagaataaaaaaaaagtaattgaagtgttgttgtcaaaaagaaaaaaaaattaagtgttttaggaaaagaagtaatcgaagtgttagcagaaaaaagaaaaaaaagttacaaattcgtatttatgaaaaaaggataaatggaatgaaattccaaagggactgtgagcagcatgcagccgtacactcgtcatggcggtggaatatgatgaaagcacggctcgtcgcgtccgacacatctctcatttcacgtgtgtctcagagtcatctgttttgtgtcgctacgtcttttctctcgactgtcttcttatagttaccatttttgttaactttaatcatttgcgtgtccatgattaatcagtctttcgaaactttaacatttctccaattcgagtgtgaccgttaatccgttatactaagatctaatcaaaacatgtctagaatcaaatttgcaatcttattgcatatttttttgtctaacaatattactagaaatctttgtttgttaccaacattagtaaaactatcatcttaaccaagttgcaggagcagttcgtttcaaacgtaattgctatagtgatgttattgtaaatttgttatactgatcaaatgtaaagaataatacaatttttatatatatcttgacaaacaaatcagtatatatatacaagaaatatagattttgtcctattacatatgcctatctcaaagttgatgtgtaaagacatgcagttcaataagccatgcaaattgagatgtgtcaaactcccttcgttaatatgtgttttcttacaatgtgaagccaaattaaattttcagaagaagacataaagatagcaactcaaatgaagtgtagattgtacatagtcgactctatatacctggttcttatctcattcaatttatcctcaaaaaactttatcaacatctatacaataagttcactataaatagcttcatctaacgcagctgtaagaccagaaaaaccacaacaactaagtaaagagaaa

SEQ ID NO: 48 - DNA, 1547 bp, Phaseolus vulgaris
  source 1..1547: mol_type = genomic DNA
gaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatggcactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctact

SEQ ID NO: 49 - DNA, 1451 bp, Glycine max
  source 1..1451: mol_type = genomic DNA
ttgacagaaaaatttaatttcctgattttagaaaagagaggaattaacggtctcaattttaaaatcttaaaactaaaaaaccaaatttatggatttgataaaataggaggatcaaaatacatttaattttttaattttttttgttttaattgattctgacagctttcaaactgtcacaaattgtaacttattttttaaatgaataatggtagtagctagggctgagcataaacccgagagaagctcgaaccaaacccacactaaaggaataaataaaaagacacaatcgggcggtttcaaattgtttattcggactatacgggtttaaaaattgcttacatggggttactttattcggatgagggtttaaaaaacttcctccggagaagaagaaagtgtttgaggccgcaaaagaaaccaagccacaacgcagattggaacacacccaagaatccgttgcattcactaagcccgaacctgacaaaatcgagaggctaatgagttagagacaatccagattattttctatcagaaatacccacagtttttaaactctgactttgtaatttgttaattgattgatatgtcattactaacctacctatgataaagagtcaaaataaattttttaagatataagaatccaatacaaagaaaaaaaaattaaaattagaactcaaaataaaattaatcatatttattatagacctaaaacatataagtatttttgtttaactaatataattgtttaaaacttagtcattttcttcttattttcgcttactttttttttatattgacaatcaaaacgaggataatccaaaaaacaaaatgtccctaatatataatttctctatccaagaaattattatatatacagtacaaattattctccactaatctttatgatatgtaaattaaagtatgattaattatttcctaattaaattaaaaaaaattgagtaactagtatcacataaagatctgtcaaaaccatgaatagtgataatttcataacacctaataatttctctatccctagagtgaggtcatttttggacggcccttcttctcactttgaggatcccatcattgtcacacggtgtattcatataatttcatatgtcataaacccgcgaacatgaaatgaaaccatggtccccctctccaccaccgttttctggcaattgcatgcaatacaaacacacttggtatttgtcacataatgttgatgtcgaactgtcgaagccacctcacacccatgaacttaatgaggtgtaacacacaaggcttccatagccatgcatactgaagaatgtctcaagctcagcaccccactcctgtgacgtgtccctcacccaccttcctctcttccctataaataaccacgcctcaggttctccgcttcacaacacaaacattctctccattgtctttgaacactcatcacc

SEQ ID NO: 50 - DNA, 1545 bp, Glycine max
  source 1..1545: mol_type = genomic DNA
ggaggtaatcctatttaagttaaataaaattagtatgaagaataattatttttaaagtatttaacttaatcgtatataaaaaattcaatatatgaataattctaagtgagtttttaagaaaaaataaaattagtaacgaagtaatttatatataattttgaaaaattatcactaaatttgtgatccactgttaacattaatttattcctcttgtattgaataaaatagttcagacatggtcccagtctttaatcaattattcatgcttctctgtctctcacttatataatcctgtaatccaaacattactcagatagctagatccaccgatcaatcgtatatatatacgcataaaatcgacgcctctgtattttttagactgtagcccaaattcactatccgaataaaataagggaggcacgtgtacgtaatttatatcatatgatagccatgcatatgcacacgtgcagaagagctgttaccctctatacgtgtactcaccttctcatcctctctgaatattttgagtgctcttcctagttatctagtaatgcatgaaattaaacttactaaatgtttcttcaatttaaagaaataattgtttatctgtttcaatttttttaagagaattttaaaaagataattgtttcggggagagagatataaaaaagaaaagggagaaatattaaaatgtactaaataatatgataagaaaagagagaaaaataaaagagaaaatttgtatatagttataattattcatgtaataaggattcatctctcaactgaaaatatacttaatgcagaagaaaaaatcattatttacaaacgttgagtcttgagtgggaaaagaggaggcgccgttactatacaatataagatcatagtactgacaaaatgcacagtaaaacagttcaaattgagaaggattcttaacacaccatagtatttaatatatatctttacagagacaattatgctggaggattcaggcaaagattatatattgtggatttgttttttaataattaacgcatcatatgaaagatcgatgatatatactaatggttataagaaaaatatttaacagtttctataacctttttcttttatcttttactgtaatattatttattttatttcacatttttaatcagcttatctcatttataaacgaaattgtataaaaatatacatgatgaactgaatagaacaatattgatctgatattctcatattgtataagaggatagactttgagacgcggagaatctgtaggaggggaccattcagagtgcctccaattttggtgttgttcattgtaccattgcaaatataaacgaagcatgcatgcttatgtatgaggtgtaacaaaattggaaacaatagccatgcaaggtgaagaatgtcacaaactcagcaacccttattcattgacgtgtccctcagtcactctcctctcatacctataaatcaccactcctcatgttctttccaattcaccaactccttcaaacttaattattaacacttccttagttcaat

SEQ ID NO: 51 - DNA, 1451 bp, Glycine max; identical to SEQ ID NO: 49
  source 1..1451: mol_type = genomic DNA

SEQ ID NO: 52 - DNA, 1454 bp, Glycine max
  source 1..1454: mol_type = genomic DNA
caactattatcgcatgatgatgtacgttaagtcatcatcatctttaactttatatattgttaaaagtagaaaaaataggtgatgcattataaaataattttataacatcatttaattataaattatttataataaatatttgagtttttatagtaattacctaaacaattatatcaagactaatgcctgattagttgacatgacgaaattaaactcataaaagtaaagatgtttatgtggaaaactcttatacaattgagcggacttttttccatggtagttcagttttcttctattcaatttatttttttggtttccgctcagaataagaataatttgataaattcatttttaggcaattaagaatatttatttgactaactttttaattgaaataaatttacaataaatactcaatttatctttcacaatcaaaagattgagatgttgtaagatctccgataatatacttatatcttttcatttattacgttttcaaatttgaattttaatgtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatcatcaagaaccagttcaatatcctagtacaccgtattaaagaatttaagatatact